(19) United States
(12) Patent Application Publication
(10) Pub. No.: US 2008/0058642 A1
(43) Pub. Date: Mar. 6, 2008

(54) METHOD FOR DETECTING CORONARY ENDOTHELIAL DYSFUNCTION AND EARLY ATHEROSCLEROSIS

(75) Inventor: K. Lance GOULD, Houston, TX (US)

Correspondence Address: CONLEY ROSE, P.C.;David A. Rose, P. O. BOX 3267, HOUSTON, TX, 77253-3267, US

(73) Assignee: BOARD OF REGENTS OF THE UNIVERSITY OF TEXAS SYSTEM, Austin, TX (US)

(21) Appl. No.: 11/848,981

(22) Filed: Aug. 31, 2007

Related U.S. Application Data
(60) Provisional application No. 60/824,509, filed on Sep. 5, 2006.

(51) Int. Cl. A61B 6/00

(52) U.S. Cl. 600/436

(57) ABSTRACT

A method of detecting endothelin receptor A mediated coronary microvascular endothelial dysfunction in an asymptomatic subject is disclosed. The method comprises obtaining sets of noninvasive cardiac PET perfusion images of the subject before and after administration of selective endothelin receptor A (ET_{A} receptor) antagonist. The images are analyzed, including application of applying Markovian homogeneity analysis, and the results are compared to detect improvement, or lack of improvement, of myocardial perfusion homogeneity in the subject. A result of improved myocardial perfusion homogeneity after administration of the antagonist indicates the presence of ET_{A} receptor-mediated microvascular endothelial dysfunction in the subject and indicates therapeutic treatment to improve endothelial function and/or to reduce coronary artery disease risk factors.

### CROSS-REFERENCE TO RELATED APPLICATIONS

This application claims the benefit under 35 U.S.C. §119(e) of U.S. Provisional Patent Application No. 60/824,509 filed Sep. 5, 2006, the disclosure of which is hereby incorporated herein by reference.

### BACKGROUND OF THE INVENTION

1. Technical Field

The present invention generally relates to diagnostic and therapeutic methods in which coronary endothelial dysfunction is imaged as resting myocardial perfusion heterogeneity; and more particularly to such methods wherein PET imaging is performed with and without selective endothelin receptor blockage.

2. Description of Related Art

Coronary endothelial dysfunction is closely associated with coronary artery disease (CAD) or its risk factors, may be familial as an independent risk factor, and predicts future coronary events or clinically manifest disease up to ten years later. The three principle methods for assessing coronary endothelial function reflect different aspects of its complex multifaceted behavior with specific limitations in clinical application. The most established method uses intracoronary acetylcholine which requires coronary arteriography and provides information only on epicardial coronary arteries, not endothelial function of the microvasculature, which is an essential component of a preclinical coronary atherosclerosis diagnosis. Secondly, forearm arterial vasodilation during reactive hyperemia by ultrasound is non-invasive but does not correlate specifically with coronary endothelial dysfunction. Cold pressor testing with measurements of coronary flow reserve involves complex sensory and efferent vasomotor control mechanisms separate from endothelial function, and there is such great variability in normal subjects that its diagnostic utility is limited.

The hallmark of coronary endothelial dysfunction is mild heterogeneous vasoconstriction of coronary arteries and/or coronary microvasculature under a wide spectrum of different conditions, and vasomotor stimuli involving many different mechanisms, including inhibition of vasodilator mechanisms or activation of vasoconstrictor mechanisms by many different interacting vasoactive mediators. Heterogeneity of coronary endothelial function has been well-documented in humans, with associated altered coronary blood flow or perfusion reflecting coronary arteriolar as well as epicardial arterial endothelial dysfunction. Resting coronary flow falls by approximately 20% after inhibition of coronary endothelial nitric oxide production without significant reduction in maximum coronary flow or coronary flow reserve measured invasively by Doppler flow velocity wires.

Coronary vascular tone depends on the balance of many simultaneously acting vasodilators and vasoconstrictors. Vascular mediators derived from coronary endothelium include prostacyclin, nitric oxide, thromboxane, endothelin-1 (ET-1), bradykinin, angiotensin, serotonin, substance P, C-type naturetic peptide (CNP, an endothelium-derived hyperpolarizing factor), and others. Nitric oxide is the primary vasodilator, while ET-1 is one of the most potent vasoconstrictors.

Endothelin exists in three isoforms, with ET-1 being the predominant form in the cardiovascular system. ET-1 exerts its effects via two receptors, type A (ET_{A}) and type B (ET_{B}). ET_{A} receptors are found predominantly on vascular smooth muscle cells and cause vasoconstriction. ET_{B} receptors have dual functions depending on their distribution. In vascular smooth muscle cells, ET_{B} receptors mediate vasoconstriction and in endothelial cells they produce vasodilatation via a nitric oxide/prostacycline pathway. In the lung, ET_{B} receptors are involved in the clearance of ET-1. Initial reports have suggested that selective ET_{A} receptor antagonists may be advantageous in heart failure with beneficial effects on survival, hemodynamics, and cardiovascular remodeling in heart failure. It has been shown that long term endothelin antagonists improved alterations in various cardiac genes in rats with heart failure, and it has been reported in the literature that the endothelin 1A receptor antagonist BSF 302146 is a potent inhibitor of neointimal and medial thickening in porcine saphenous vein-carotid artery interposition grafts.

Selective endothelin receptor antagonists (ERAs) are also of value in hypertension. Essential hypertension has been linked to endothelial dysfunction. ET-1 not only raises blood pressure but also causes vascular and myocardial hypertrophy. Endothelin blockade has also been shown to decrease blood pressure without causing a change in heart rate and to prevent vascular hypertrophy. Experimentally, selective ET_{A}-receptor antagonists prevent endothelial vasomotor dysfunction in atherosclerosis. Selective ET_{A}-receptor antagonists have also been shown to improve flow-mediated vasodilatation in heart failure. While a recent randomized trial of the ET_{A}-receptor antagonist Darusentan™ in heart failure showed no benefit, the powerful vasoconstrictive effects of ET-1 are well-documented and its pathophysiological role remains an important question.

Since coronary endothelial dysfunction is associated with preclinical coronary atherosclerosis, preceding the earliest stages of coronary artery disease in association with increased risk of coronary events and subsequent clinically manifest coronary artery disease many years later, early detection and quantitative assessment of coronary endothelial dysfunction is of great potential value in providing a basis for intense, lifelong, pharmacologic and lifestyle preventive treatment. While coronary flow reserve and myocardial perfusion imaging after pharmacologic arteriolar vasodilation for identifying flow-limiting coronary artery stenosis is now widespread as a routine clinical diagnostic procedure, there currently exists only limited data on PET scan results as a marker of coronary endothelial function. There is continuing interest in development of ways to detect early preclinical atherosclerosis.

### SUMMARY OF THE INVENTION

In accordance with certain embodiments of the invention, a method of detecting endothelin receptor A (ET_{A} receptor) mediated microvascular coronary endothelial dysfunction in an asymptomatic subject is provided which comprises: (a) obtaining a first set of noninvasive cardiac PET perfusion images of the subject; (b) analyzing the cardiac PET perfusion images obtained in step (a), wherein analysis of the images comprises applying Markovian homogeneity analysis to the first set of images, to yield a first result comprising an initial myocardial perfusion homogeneity index; (c) administering at least one selective endothelin receptor A antagonist to the subject; (d) obtaining a second set of noninvasive cardiac PET perfusion images of the subject after the administration of the antagonist; (e) analyzing the cardiac PET perfusion images obtained in step (d), wherein analysis of the images comprises applying Markovian homogeneity analysis to the second set of images, to yield a second result comprising a second myocardial perfusion homogeneity index; and (f) comparing the first and second results to detect improvement of myocardial perfusion homogeneity, or lack thereof, in the subject, wherein a result of improved myocardial perfusion homogeneity after administration of the antagonist indicates the presence of endothelin receptor A mediated microvascular coronary endothelial dysfunction in the subject.

In certain embodiments, the method aids in diagnosing early coronary artery disease in an asymptomatic subject at risk of developing coronary artery disease. “Early coronary artery disease” is also called coronary atherosclerosis with no clinical manifestations, i.e., absence of heart attack, chest pain, bypass surgery, balloon dilations or stents and/or severe flow-limiting stenosis. “Asymptomatic” refers to the absence of heart attack, chest pain, bypass surgery, balloon dilation, stents, heart failure or any other overt symptoms of coronary artery disease.

In certain embodiments, steps (a) and (d) further comprise administering a pharmacologic cardiac stress agent prior to obtaining the PET images. In some embodiments, the cardiac stress agent comprises dipyridamole or adenosine, administered by intravenous infusion.

In certain embodiments, the method further comprises (g) comparing Markovian homogeneity analyses of cardiac PET perfusion images obtained with and without administration of the stress agent to the subject, wherein a result of improved myocardial perfusion homogeneity during pharmacologically induced cardiac stress or after endothelin blockers at rest or stress is indicative of an abnormality of endothelial dysfunction. Thus, in some embodiments, PET images are obtained at rest and after dipyridamole or adenosine stress before and after giving the endothelin blocker to demonstrate the effects of the endothelin blocker on rest and stress coronary blood flow. In some embodiments, at least one ET_{A}-receptor antagonist is selected from the group consisting of Darusentan™, Sitaxsentan™, BQ123, BMS1822874, PD156707TTA101, 34-sulfatobastadin and BSF302146.

In some embodiments, improvement of myocardial perfusion homogeneity is quantitated at least in part by Markovian homogeneity analysis of cardiac PET perfusion images of the subject. In some embodiments, the cardiac PET perfusion images comprise resting images.

In certain embodiments, the subject, prior to administering the endothelin receptor A antagonist, exhibits a baseline resting myocardial perfusion homogeneity expressed as a Markovian homogeneity number that is outside about 2 standard deviation limits of a mean Markovian homogeneity number of a control group of normal healthy subjects. In some embodiments, following administration of the endothelin receptor A antagonist, an increase in the Markovian homogeneity number is obtained.

In certain embodiments, the analysis of cardiac PET perfusion images comprises detection of regional perfusion defects, if any. In some embodiments, in (f), the comparison indicates a reduction in the size and/or severity of regional perfusion defects following administration of the endothelin receptor A antagonist.

In certain embodiments, in steps (b) and (e), the analysis of cardiac PET perfusion images comprises Markovian homogeneity analysis and either (i) observation of regional perfusion defects, or (ii) measurement of a base to apex longitudinal perfusion gradient, or both (i) and (ii). In some embodiments, the gradient is reduced following administration of the endothelin receptor A antagonist.

In certain embodiments, in step (b), the analysis of the cardiac PET perfusion images reveals that an abnormal baseline resting myocardial perfusion homogeneity expressed as a Markovian homogeneity number that is lower than the mean Markovian homogeneity number of a control group of healthy subjects by a margin greater than about 2 standard deviation limits.

In certain embodiments, in step (f), a result of no improvement of myocardial perfusion homogeneity after the administration of the ET_{A}-receptor antagonist indicates the absence of ET_{A}-mediated microvascular endothelial dysfunction in the subject. In some embodiments, the indication of an absence of endothelin receptor A mediated microvascular endothelial dysfunction in the subject is diagnostic of early coronary artery disease. In some embodiments, the diagnosis of early coronary artery disease indicates therapeutic treatment of the subject to improve coronary endothelial function and/or to reduce coronary artery disease risk factors. In some embodiments, one kind of therapeutic treatment comprises administration of a myocardial perfusion homogeneity enhancing amount of at least one endothelin receptor A antagonist. For example, Darusentan™ may be administered in a daily dose of about 1 to about 600 mg, preferably about 5 to about 300 mg, and more preferably about 50 to about 150 mg. The Darusentan may be administered for a period of at least about 1 week, administered once or more daily.

In certain embodiments, the subject has one or more risk factors associated with coronary disease. In certain embodiments, an indication of the presence of endothelin receptor A mediated microvascular endothelial dysfunction is diagnostic of existing coronary atherosclerosis and/or elevated risk of future coronary artery disease in the subject.

Also provided in accordance with certain embodiments of the present invention is a method for reducing incidence or risk of a disease or adverse event related to coronary endothelial dysfunction in a subject who has been determined to have endothelin receptor A mediated microvascular coronary endothelial dysfunction, by using an above-described method of detection. In some embodiments, the therapeutic method comprises administering to the subject a myocardial perfusion homogeneity enhancing amount of one or more ET_{A}-receptor antagonist effective to alleviate ET_{A}-mediated microvascular endothelial dysfunction and enhance myocardial perfusion homogeneity in the subject. Alternatively, or additionally, another therapy is administered to the subject to ameliorate ET_{A}-mediated microvascular endothelial dysfunction or decrease coronary artery disease risk factors in the subject. In certain embodiments, the therapeutic method is effective to deter the onset or severity of coronary atherosclerosis, coronary artery disease, myocardial ischemia, angina, microvascular angina, myocardial infarction, sudden cardiac death, arrhythmia, heart failure, dilated cardiomyopathy, cardiac dysfunction, pulmonary embolism or cardiogenic shock, or any combination of those conditions. In some embodiments, the analysis of cardiac PET perfusion images occurs before, and at least once during and/or after the period of ET_{A}-receptor antagonist administration.

These and other embodiments, features and advantages will be apparent from the following description and drawings.

### BRIEF DESCRIPTION OF THE DRAWINGS

FIG. 1 is a schematic showing four topographic views of myocardial perfusion by PET. From left to right, views are left, inferior, right or septal, and anterior. Each view corresponds to the distribution of coronary arteries shown.

FIG. 2 is a black and white rendering of a set of color PET images of individuals at rest and after dipyridamole stress in three representative clinical examples. White and grey shades indicate normal or adequate myocardial perfusion. Dark shades of grey or black indicate reduced myocardial perfusion due to coronary artery disease (black arrows). Panel A: young healthy normal volunteer without risk factors for vascular disease. Panel B: a subject with severe stress-induced perfusion defect.

FIG. 3 is a display of the relative activity distribution for Markovian homogeneity analysis, wherein each square panel corresponds to quadrants of the same subjects as in FIG. 2. Quadrant squares, corresponding to the views shown at the bottom of FIG. 1, indicate the area of heterogeneity analysis with the basal slices and apex excluded from analysis. Dark areas indicate lower flow areas causing heterogeneity on the PET perfusion images.

FIG. 4 is two graphs of base-to-apex longitudinal perfusion gradient expressed as first derivative or spatial slope of relative activity (vertical axis) at each tomographic slice from base-to-apex (horizontal axis) at rest (diamonds) and with dipyridamol stress (Xs) with +2 SD and −2 SD limits of 50 reference subjects at rest (dashed lines labeled “R”) and after dipyridamole (dashed lines labeled “D”)

FIG. 5 is a bar graph of relative percentage distribution of subjects in ±2 SD (standard deviations) from normals of relative radionuclide uptake distribution, corresponding to data obtained from representative groups of patient with abnormal, borderline or normal myocardial perfusion homogeneity.

FIG. 6 a schematic diagram of a protocol for demonstrating the efficacy of an endothelin receptor A antagonist in improving myocardial perfusion homogeneity.

FIG. 7 is a schematic diagram of an experimental protocol to demonstrate endothelin-induced myocardial perfusion defects. IV, intravenous; IC, intracoronary.

FIG. 8 is a group of PET images showing resting endothelin-induced resting perfusion defect, with improvement after intravenous adenosine in accordance with the protocol of FIG. 7. Panels A: vertical long-axis views. Panels B: short-axis views. AD, adenosine; ET, endothelin-1. Arrows indicate the region of endothelin-induced perfusion defect. iv, intravenous; ic, intracoronary.

FIG. 9 is a group of PET images showing resting endothelin-induced resting perfusion defect, with worsening after intravenous adenosine. Panels A: vertical long-axis view. Panels B: short-axis views.

FIG. 10 is a graph showing relative ⁸²Rb uptake expressed as % of baseline left circumflex coronary artery (LCx) uptake for the same pixel of each image of the protocol illustrated in FIG. 7. Bars=±SD.

FIG. 11 is a graph showing relative ⁸²Rb uptake expressed as % of baseline LCx uptake for the same pixel of each image of the protocol of FIG. 7. For group 1, improvement after intravenous adenosine is shown. For group 2, no improvement after intravenous adenosine is shown. Bars=±SD.

### DETAILED DESCRIPTION

The visually apparent heterogeneity of resting myocardial perfusion and/or its improvement after dipyridamole or adenosine stress on high quality, non-invasive PET images is thought to be one manifestation of coronary arteriolar endothelial dysfunction. Positron emission tomography (PET) is particularly suited for imaging this pattern of heterogeneous perfusion without the attenuation artifacts and poor depth dependent resolution of standard single photon emission tomography (SPECT). Gould et al. first reported the concept of (i) coronary flow reserve, (ii) pharmacologic stress perfusion imaging, and (iii) improved myocardial perfusion after intense lipid treatment in CAD. The application of Markovian Homogeneity Analysis to myocardial PET perfusion images provides the first automated, objective methodology for precisely quantifying visual heterogeneous perfusion patterns, such as those which have been previously described. It is believed that there is no prior PET scan study that includes administration of an ET_{A}-receptor antagonist.

### EXAMPLES

### Example 1

### Clinical Evaluation of Myocardial Perfusion Heterogeneity Quantified by Markovian Analysis of PET Images

In this paradigm, the resting perfusion image serves as a baseline for comparison with the stress perfusion image for identifying discrete regional perfusion abnormalities due to flow-limiting coronary artery stenosis, myocardial scar, or hibernating myocardium. The present example analyzes and quantifies the distinctly different diffuse patchy heterogeneity of resting myocardial perfusion as a marker of coronary endothelial dysfunction associated with coronary atherosclerosis, independently from and around these traditional discrete regional myocardial perfusion defects caused by flow-limiting stenosis or myocardial scar.

A mathematic technique from Markovian homogeneity analysis is used to provide precise, objective, automated quantification of resting perfusion heterogeneity in 1,034 subjects, its normal limits in 50 healthy reference subjects, and its close association with documented CAD, thereby demonstrating a basic new observation in myocardial perfusion imaging with potentially important clinical implications. This work is also described in the publication of Johnson N P and Gould K L, “Clinical evaluation of a new concept: resting myocardial perfusion heterogeneity quantified by markovian analysis of PET identifies coronary endothelial dysfunction and early atherosclerosis in 1,034 subjects.” J Nucl Med 46: 1427-1437, 2005, the disclosure of which is hereby incorporated herein by reference.

### Materials and Methods

Study Patients. The population for this study consists of 1,034 consecutive subjects undergoing diagnostic, rest-dipyridamole, myocardial perfusion PET at The Weatherhead PET Center For Preventing and Reversing Atherosclerosis of the University of Texas Medical School—Houston. All subjects signed informed consent approved by the Committee for the Protection of Human Subjects of the University of Texas Health Science Center. A complete medical history was obtained on all patients undergoing diagnostic cardiac PET for assessment or follow-up of known CAD, for second opinions on revascularization procedures, for prior positive stress tests, for coronary calcification by CT, for chest pain or other symptoms, for screening, or for risk factors. A history of risk factors was obtained for age, sex, diabetes, hypertension, high cholesterol, family history of vascular disease, excess weight, lack of exercise, and past or present smoking, that were counted as positive even if treated, as with hypertensive or lipid-lowering medications.

PET. Patients were instructed to fast for 4 h and abstain from caffeine, theophylline, and cigarettes for 24 h before study. PET was performed using the University of Texas designed, Positron Posicam Auricle, bismuth germanate, 2-dimensional (2D) multislice tomograph with a reconstructed resolution of 10-mm full width at half maximum. Using a rotating rod source containing 148-185 MBq (4-5 mCi) of ⁶⁸Ge, transmission images to correct for photon attenuation contained approximately 40-60 million counts. Emission images obtained after intravenous injection of 925-1,850 MBq (25-50 mCi) of generator-produced ⁸²Rb contained 20-50 million counts depending on the age of the generator and size of the patient. After resting ⁸²Rb data acquisition, dipyridamole (0.142 mg/kg/min) was infused for 4 min. At 4 min after completion of the dipyridamole infusion, the same dose of ⁸²Rb was given intravenously.

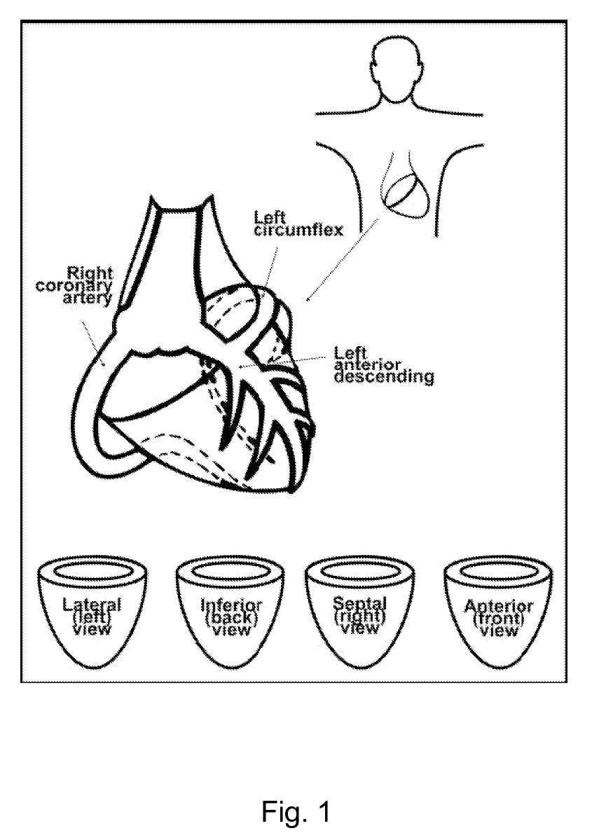

Automated Quantitative Analysis of PET Images. Completely automated analysis of the severity and size of PET abnormalities was performed by previously described software. For example, Gould et al., Circulation 101:1931-1939, 2000. A 3-dimensional (3D) restructuring algorithm generates true short- and long-axis views from PET transaxial cardiac images acquired in 2D tomographic mode to minimize scatter. From circumferential profiles, 3D topographic views of the left ventricle are reconstructed showing relative regional activity distribution divided into lateral, inferior, septal, and anterior quadrant views of the 3D topographic display corresponding to the coronary arteries illustrated in FIG. 1.

Each topographic map consists of 21 slices along the long axis of the left ventricle. Every long-axis slice contains 64 radial pixels, representing equal angles around a circle (360° divided over 64 pixels equals just under 6° per pixel). The 4 quadrant views contain 16 radial pixels each. Therefore, the absolute pixel size is N-by-M, where N is 1/21 of the base-to-apex distance (different for every patient) and M is 360°/64° (same for every patient).

Activity is normalized to the maximum 2% of pixels in the whole heart dataset. Regions of each quadrant are identified as outside 97.5% confidence intervals (CI) or 2.5 SD of 50 healthy control subjects with no risk factors by complete medical history. The percentage of circumferential profile units outside 97.5% CI is calculated automatically after correcting for any misregistration of attenuation and emission images that commonly cause artifactual defects.

Markovian Homogeneity Analysis. Markovian texture or homogeneity analysis characterizes an image by examining the probability that a pixel with a given intensity will have a neighbor with a different intensity, where Pd(m) is the probability that 2 adjacent pixels have intensity values that differ by m. For the purposes of this disclosure, the homogeneity index, H, is given by the following equation:

H=Σₘ[1/(1+m)²]Pd(m) Eq. 1

The homogeneity index, H, can have values between 0 (noninclusive) and 1 (inclusive). A value near 0 represents an image with a high probability that neighboring pixels have intensity values that differ greatly. The homogeneity index cannot be 0 because at least one Pd(m) must be nonzero. A large value near 1 represents an image with a high probability that neighboring pixels have similar intensity values. In principle, the homogeneity index can be 1, in which case all pixels have the same intensity.

The homogeneity index thus quantifies mathematically the intuitive notion of homogeneity. A perfusion image that is inhomogeneous or diffusely patchy has a small homogeneity index near 0, whereas a uniform image has a large index near 1. Conversely, an image with a small homogeneity index can be considered to be heterogeneous and vice versa. Each pixel can have a maximum of 8 neighbors: above, below, left, right, above left, above right, below left, and below right, where a complete topographic map is like the surface of a cylinder and “wraps” along the radial dimension. There are 5,184 unique pixel pairs for a 21×64 matrix, assuming the 64-axis wraps. Intensity of the image matrix is normalized to 1,000. More generally, for an N-by-M matrix, assuming the M-axis wraps, there are (4 NM−3M) unique pixel pairs. Radial pixel size and, hence, heart size does not impact the calculation of the heterogeneity index.

Equation 1 shows that as the differences among neighboring pixel units become larger for severe defects, the coefficient 1/(1+m)² decreases rapidly with increasing m, corresponding to increasing differences of intensity between neighboring pixels. Expanding the summation in Equation 1 for differences of m=0, 1, 2, 3, etc., yields the following:

Homogeneity Index=Pd(0)+¼Pd(1)+ 1/9Pd(2)+ 1/16Pd(3)+. . .

This expansion shows that for a difference of 2 between neighboring pixel units, the contribution of the corresponding component, Pd(2), contributes just over 11% as much as Pd(0) to the homogeneity index. Therefore, the homogeneity index, H, expresses as a single number the probability distribution of differences among neighboring pixel units that is weighted for small differences among neighboring pixel units with little influence on H by large differences due to severe discrete regional defects. The value of H therefore objectively quantifies the extent of the mild diffuse heterogeneous patchy pattern on PET images separately from, independent of, and around more severe discrete regional perfusion defects caused by flow-limiting stenosis.

Application of Homogeneity Analysis to PET Perfusion Images. The rest and stress scans are displayed as topographic displays in 4 quadrant views (lateral, inferior, septal, and anterior). For applying Equation 1 to this topographic map, 3 modifications were made as follows: (i) The basal 4 slices are discarded to avoid count variability in the membranous septum and the apical 2 slices are discarded to minimize partial-volume effects and variability in locating the last apical slice; (ii) pixels with intensity values below 500 are reset to 500, and pixels with intensity values above 850 are reset to 850 to eliminate any effect on the homogeneity index, H, of very low activity levels of myocardium scar and to eliminate effects of the highest activity levels of normal myocardium.

In effect, these limits further confine the homogeneity analysis to relative activity values ranging from 50% to 85% of maximum on each PET image, thereby excluding extreme values as from severe defects or hot spots that would bias the value of H for quantifying more subtle differences among pixel units; (iii) these modified intensity values, 500-850 inclusive, are scaled into an integer range of 35 levels, so that each new intensity level represents 1% of the range, thereby mathematically further restricting the analysis to myocardium that is not scarred, severely ischemic, or maximally perfused. Consequently, the homogeneity index is not greatly influenced by severe perfusion defects. The degree of small-scale diffuse heterogeneous “patchiness” is objectively quantified on resting and stress images and the rest-to-stress change independently of, separately from, or around severe discrete regional perfusion defects due to myocardial scar, reduced coronary flow reserve of flow-limiting stenosis, or the base-to-apex longitudinal perfusion gradient due to diffuse disease. The scaling impacts the heterogeneity index and determines the “coarseness” or the degree of patchiness being quantified.

Mean and SD values for the homogeneity index, H, were computed for the 50 healthy control subjects just as for the other automated quantitative measurements on the PET images for comparison with the patients.

Statistical Analysis. All statistical analyses were performed using SPSS version 11.5 (SPSS Inc.). Data are reported as mean±SD or SEM as appropriate.

Analysis 1. Multivariate logistic regression analysis was performed with the independent variables being the continuous values of the resting homogeneity index (rH), the rest-to-stress change in H (rsHΔ), and all discrete risk factors of age, sex, history of diabetes, hypertension, high cholesterol, family history of vascular disease, excess weight, smoking, menopausal status, and lack of exercise. The dependent variables were an abnormal PET after dipyridamole (stress PET) defined as either the lowest mean quadrant activity on the stress PET image, Q, outside 1 SD of healthy reference subjects (Q<1 SD), indicating flow-limiting stenosis, or the base-to-apex longitudinal perfusion gradient (L) after dipyridamole outside 1 SD of healthy reference subjects (L<1 SD), indicating diffuse coronary artery narrowing. An abnormal stress PET therefore includes all cases with any abnormality of either Q or L and excludes those with completely normal Q and L. That is, both Q>1 SD and L>1 SD. It indicates a not-normal PET perfusion scan after dipyridamole stress attributed to either a localized regional defect or an abnormal base-to-apex longitudinal perfusion abnormality outside 1 SD of healthy reference subjects, thereby objectively documenting even mild CAD.

Analysis 2. Multivariate linear regression analysis was performed with the same independent variables as above. That is, the continuous values of the resting homogeneity index (rH), the rest-to-stress change in H (rsHΔ), and all discrete risk factors. The dependent variable is the continuous value of the lowest mean quadrant activity of the stress PET image (Q), indicating the quantitative severity of regional perfusion defects after dipyridamole caused by flow-limiting stenosis.

A Pearson X² analysis was performed for the discrete variables as follows: abnormal homogeneity (rH<2 SD or rsHΔ<2 SD), borderline homogeneity (rH and rsHΔ within 1-2 SD), normal homogeneity (rH>1 SD and rsHΔ>1 SD), abnormal stress scans (Q<2 SD or L<2 SD), borderline stress scans (Q and L within 1-2 SD), and normal stress scans (Q>1 SD and L>1 SD). A 2-tailed P value<0.05 was considered statistically significant.

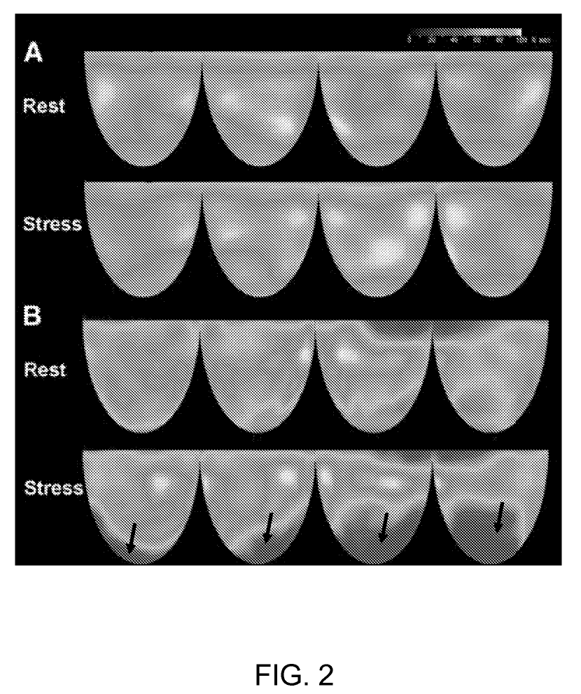

Results. Complete data on 1,034 patients were analyzed. FIG. 1 illustrates orientation of PET perfusion images in lateral, inferior, right, and anterior topographic views. FIG. 2 shows 2 examples of rest-dipyridamole PET illustrating the range of images and quantitative measurements of the homogeneity index, the severity of stress-induced regional perfusion defects caused by flow-limiting stenosis, and the base-to-apex longitudinal perfusion gradient due to diffuse coronary atherosclerosis. The first pair of rest-stress images (FIG. 2, Panel A) are of a young healthy volunteer with no coronary risk factors as an example of normal perfusion images. The second rest-stress pair (FIG. 2, Panel B) is from a patient with a severe stress-induced perfusion defect in the distribution of the mid left anterior descending coronary artery. This example of a severe stress-induced perfusion defect illustrates that the Markovian homogeneity analysis is independent of and separate from even severe perfusion defects because it improves from a low value of 0.34 at rest that is >2 SD of the healthy reference group to 0.49 after dipyridamole, within 1 SD of normal, despite a severe stress-induced perfusion defect.

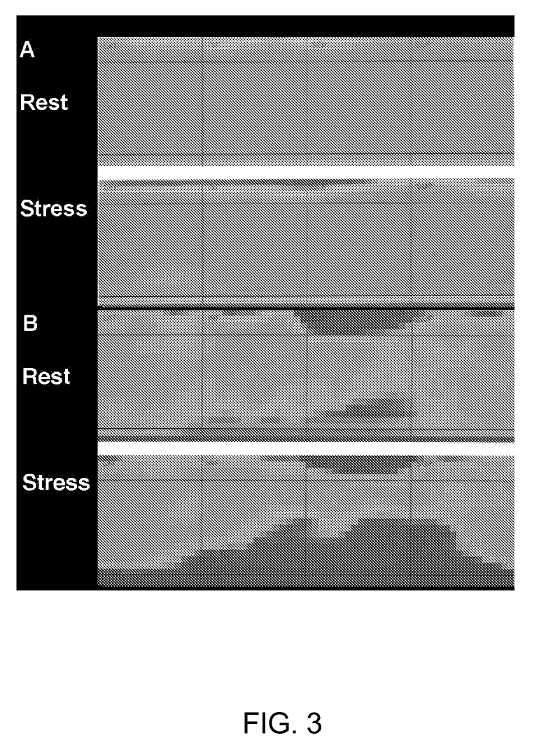

FIG. 3 shows the corresponding graphic displays of homogeneity analysis for these same two examples in the same order. For the first rest-stress pair (FIG. 3, Panel A), the homogeneity index by Markovian analysis is 0.80 at resting conditions and remains comparable at 0.83 after dipyridamole, both within the normal limits of 50 healthy control subjects. The second rest-stress pair of the person with a severe stress-induced perfusion defect, FIG. 3, Panel B shows severe resting perfusion heterogeneity with a homogeneity index of 0.34 that is >2 SD of healthy reference subjects and improves after dipyridamole to 0.49, within 1 SD of healthy reference subjects, in regions around the severe stress-induced perfusion defect due to flow-limiting coronary artery stenosis. As a quantitative measure of the stress-induced perfusion defect, the lowest mean average quadrant activity on the dipyridamole scan is 63% of maximum that is >7 SD of healthy reference subjects and the base-to-apex longitudinal perfusion gradient is <5 SD of normal limits. This example illustrates that the homogeneity index quantifies the patchy heterogeneous perfusion pattern separately from, independently of, and around localized regional perfusion defects.

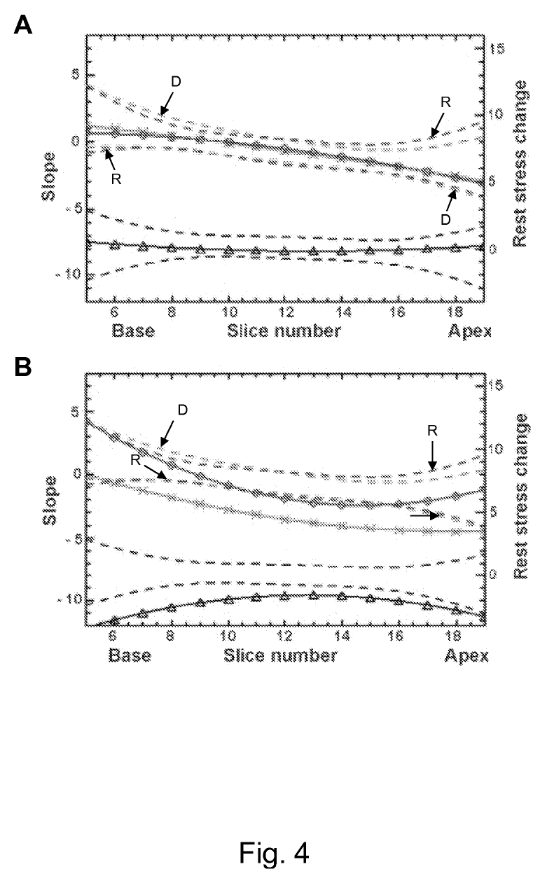

FIG. 4 illustrates the base-to-apex longitudinal perfusion gradient of these same two pairs of rest-stress PET scans, being within normal limits for the first pair (FIG. 4, Panel A) and markedly abnormal for the other example (FIG. 4, Panel B), indicating diffuse CAD plus a severe localized flowlimiting stenosis (FIG. 4, Panel B). In FIG. 4, slope units are changes in relative activity per base-to-apex. For the healthy control person (A), the base-to-apex longitudinal perfusion gradient at rest and during dipyridamole stress and the rest-to-stress change are both within 2 SD of 50 healthy control subjects. For the person with the severe stress-induced perfusion defect (B), the longitudinal perfusion gradient at rest and stress and the rest-to-stress change are all outside 2 SD of healthy reference subjects.

Table 1 summarizes the logistic regression analysis for the resting homogeneity index (H), its rest-to-stress improvement (rsHΔ), and the risk factors as the independent variables. The discrete dependent variable is any abnormality of the stress perfusion scan, either the minimum quadrant average activity outside, or greater than, Q>1 SD of healthy reference subjects or the base-to-apex longitudinal perfusion gradient outside, or greater than, L>1 SD of healthy reference subjects on stress PET images. As expected, standard risk factors are predictive of abnormal stress perfusion images. A family history of vascular disease and smoking were not significantly predictive because of the brevity of details of the history recorded in the database options. The family history did not differentiate among parents, siblings, or remote relations. Smoking did not differentiate among remote brief smoking, active current smoking, or amount of smoking.

TABLE 1
Multivariate Stepwise Logistic Regression Analysis For Any Stress Induced Myocardial
Perfusion Abnormality By PET Imaging As The Dependent Variable

| | Dependent | | | | |
|---|---|---|---|---|---|
| | variable | | | | |
| | Discrete: any | | | 95% CI of | P for |
| Independent variable | abnor | B | EXP(B) | EXP(B) | significance |
| Resting Homogeneity Index, rH | Q < 1SD or L < 1SD | −23.1 | 0.000 | .000-.000 | <0.001 |
| Rest to stress change in H, | Q < 1SD or L < 1SD | −22.2 | 0.000 | .000-.000 | <0.001 |
| rsHΔ | | | | | |
| Male | Q < 1SD or L < 1SD | 1.99 | 7.29 | 3.40-15.63 | <0.001 |
| Age | Q < 1SD or L < 1SD | 0.02 | 1.02 | 1.00-1.04 | 0.019 |
| Hx of diabetes | Q < 1SD or L < 1SD | 1.88 | 6.57 | 2.39-18.03 | <0.001 |
| Hx of hypertension | Q < 1SD or L < 1SD | 0.55 | 1.73 | 1.29-2.33 | <0.001 |
| Hx of overweight | Q < 1SD or L < 1SD | 0.43 | 1.53 | 1.13-2.07 | <0.001 |
| Hx of exercise | Q < 1SD or L < 1SD | 0.57 | 1.77 | 1.04-3.02 | 0.036 |
| Hx of high cholesterol | Q < 2SD | 0.85 | 2.33 | 1.32-4.11 | 0.004 |
| Postmenopausal | Q < 2SD | 3.02 | 20.45 | 1.98-288.2 | 0.011 |
| Family Hx vascular disease | Q < 2SD | −0.18 | 0.84 | 0.48-1.47 | NS |
| Hx of smoking | Q < 2SD | 0.06 | 1.07 | 0.78-1.45 | NS |

B = logₑ of the odds ratio.
CI = confidence interval.
EXP = exponent.
SD = standard deviation.
Hx = history of.
Q = lowest average quadrant activity on stress PET image
L = longitudinal base to apex perfusion gradient on stress PET image

The resting homogeneity index and its rest-to-stress change are powerful predictors of stress-induced perfusion abnormalities separately from and independently of standard risk factors. The much larger values of B in the regression equation indicate that resting heterogeneity and its rest-to-stress improvement are not only independent of but also markedly more powerful predictors of stress-induced myocardial perfusion abnormalities than standard risk factors.

The negative values of B for the homogeneity index (rH) and its rest-to-stress change (rsHΔ) indicate that high values of rH or rsHΔ are associated with very high probability of normal stress perfusion images (the minimum quadrant average activity Q less than or within 1 SD of healthy reference subjects) and a low probability of abnormal stress perfusion images (Q greater than or outside 1 SD of healthy reference subjects). Similarly, low values of rH or its rest-to-stress change, rsHΔ, are associated with a very high probability of abnormal stress perfusion defects (Q>1 SD) and a low probability of normal stress perfusion images (Q<1 SD).

For logistic regression, which uses a sigmoid/logistic model instead of a linear one, B is the logarithm of the odds ratio, and EXP(B) is the ratio of the odds. That is, the probability of something occurring divided by the probability of something not occurring, quantified as EXP(B). For example, in this analysis for diabetes, B is 1.64, the EXP(B) is e^{1.64} or 5.15. That is, the odds ratio for diabetes. Therefore, a person with a history of diabetes carries a 5.15 times greater odds of an abnormal stress PET scan than the odds for a person without a history of diabetes.

For the homogeneity index, B is −23.05, the EXP(B) is e^{-23.05} or 1.03×10⁻¹⁰. That is, the odds ratio for the homogeneity index. Therefore, the odds of a patient with a normal homogeneity index (HI=1.0) having an abnormal stress PET scan is only 0.000000000103 of the odds for a patient with an abnormal homogeneity index (HI=0.0+) of having an abnormal stress PET. Similarly, the odds for a patient with an abnormal homogeneity index of having a normal stress image is an equally small percent of the odds for a patient with a normal homogeneity index of having a normal stress image. From the other viewpoint, the odds for a person with an abnormal resting homogeneity index (HI 0.0+) of having an abnormal stress PET is 1/0.0000000103 or 9.7×10⁹ times the odds of a person with a normal homogeneity index (HI+1.0) of having an abnormal stress PET. Separately and independently of the resting homogeneity index, rH, the rest-to-stress improvement in the homogeneity index, rsHΔ, after dipyridamole stress is comparably predictive of CAD with a comparable odds ratio.

Table 2 summarizes the multivariate linear regression analysis with the independent variables being the resting perfusion homogeneity index (rH), the rest-stress change in homogeneity index (rsHΔ), and all risk factors together in the first 5 rows and for the risk factor alone without the PET data in rows 7 through 17. The single dependent variable is the continuous quantitative severity of stress perfusion defects. That is, the minimum average quadrant activity on the stress PET images (Q). This analysis shows that the resting homogeneity index and its rest-to-stress improvement are closely correlated with stress-induced regional perfusion defects separately from and independently of other risk factors (P<0.001). For linear regression analysis of continuous variables, B is the “slope” of the regression equation (coefficient of the independent variable in the fitted equation) that is substantially greater for the resting homogeneity index and its rest-to-stress change than for any of the standard risk factors.

TABLE 2
Multivariate Stepwise Linear Regression Analysis for Severity of Stress
Induced Myocardial Perfusion Abnormality as theContinuous Dependent
Variable (Q

| | | P for |
|---|---|---|
| | B | significance |
| Independent variable, all | | |
| Risk factors and PET images | | |
| Resting Homogeneity Index, rH | 38.0 | <0.001 |
| Rest to stress change in H, rsHΔ | 34.3 | <0.001 |
| Hx of Diabetes | −3.31 | <0.001 |
| Age | −0.07 | <0.001 |
| Independent variable: | | |
| Risk factors only | | |
| Male | −5.71 | <0.001 |
| Age | −0.11 | <0.001 |
| Hx of diabetes | −4.74 | <0.001 |
| Hx of overweight | −1.86 | 0.001 |
| Hx of hypertension | −1.34 | 0.016 |
| Hx of smoking | −1.13 | 0.033 |
| Family Hx vascular disease | 0.49 | NS |
| Postmenopausal | −0.75 | NS |
| Hx of exercise | −0.48 | NS |
| Hx of high cholesterol | −1.11 | NS |

Q = lowest average quadrant activity on stress PET
B = slope of the linear regression

The negative values of B for the risk factors indicate that the standard risk factors are associated with more severe stress-induced perfusion abnormalities. That is, lower values of the minimum quadrant average activity, Q. The positive values of B for homogeneity index (H) and its rest-to-stress change indicate that low values of rH. That is, more heterogeneous resting perfusion images are associated with more severe stress-induced perfusion abnormalities. That is, lower values of the minimum average quadrant activity, Q. Similarly high values of rH are associated with less severe stress-induced perfusion abnormalities or normal images. That is, high values of Q. For linear regression, the odds ratios based on B are not applicable. Quantitative severity of the stress perfusion defects, Q, was not predicted by family history of vascular disease, smoking, postmenopausal status, or history of high cholesterol, again, most likely due to the brevity of the history details that also did not account for cholesterol levels or its treatment.

Table 3 shows the X² analysis with numbers of subjects in each category where homogeneity was defined as “abnormal” if either the resting homogeneity index or its rest-to-stress improvement were outside or <2 SD of healthy reference subjects, “normal” if both were >2 SD, and “borderline” for all other combinations of the rest and rest-to-stress change as mixed 1-2 SD, <2 SD, and >2 SD. Similarly, stress images were defined as “abnormal” if either the lowest mean quadrant average activity (Q) caused by flow-limiting stenosis or the longitudinal base-to-apex perfusion gradient (L) due to diffuse CAD were <2 SD of healthy reference subjects, normal if both were >2 SD, and borderline for all other combinations. In Table 3, the distribution of subjects in the binary discrete categories of homogeneity and stress perfusion categories is significant with P<0.001.

TABLE 3
Number of Patients in Chi Square Analysis

| | Stress | Stress | | |
|---|---|---|---|---|
| | Abnormal | Borderline | Stress Normal | Total |
| Homogeneity Abnormal | 171 | 22 | 16 | 209 |
| Homogeneity Borderline | 316 | 90 | 127 | 533 |
| Homogeneity Normal | 72 | 38 | 182 | 292 |
| Total | 559 | 150 | 325 | 1034 |

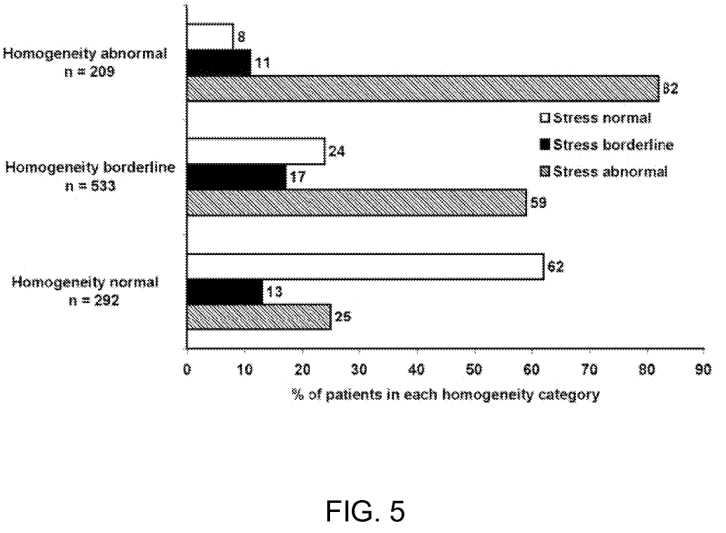

Table 4 shows the relative or percentage distribution of the X² distribution of raw numbers in Table 3 expressed in Table 4 as the percentage of the patients in each homogeneity category in rows from left to right across the table. FIG. 5 is a bar graph of the relative percentage distribution of patients in each of the homogeneity categories derived from the X² analysis in Table 3 and the percentage distributions in Table 4.

TABLE 4
Percent Distribution of Patients in Each Homogeneity Group

| | Stress | Stress | Stress | | |
|---|---|---|---|---|---|
| | Abnormal | Borderline | Normal | N | |
| Homogeneity Abnormal | 82% | 11% | 8% | 209 | 100% |
| Homogeneity Borderlne | 59% | 17% | 24% | 533 | 100% |
| Homogeneity Normal | 25% | 13% | 62% | 292 | 100% |

Table 5 shows the mean values of the homogeneity index (rH), the severity of the stress perfusion defect (Q) and the longitudinal perfusion gradient (L) for all patients grouped according to a binary classification based on the homogeneity index being >2 SD, 1-2 SD, <1 SD for comparison with the mean values for the healthy control subjects. Mean values in all the categories are significantly different from healthy control subjects with P<0.001 and are different from each other by ANOVA with P<0.001.

TABLE 5
Mean Values Of Quantitative Endpoints

| Binary rH | Resting | | | |
|---|---|---|---|---|
| group | Homogeneity Index, rH | Q | L | n |
| <2SD | 0.32 ± 0.036* | 70.9 ± 9.1* | 1.61 ± 1.85* | 157 |
| 1-2SD | 0.44 ± 0.035* | 73.3 ± 9.6* | 0.85 ± 1.44* | 388 |
| >1SD | 0.61 ± 0.079* | 77.6 ± 7.4* | 0.66 ± 1.24* | 439 |
| normals | 0.63 ± 0.127 | 82.3 ± 2.8 | 0.09 ± 0.45 | 50 |

SD = standard deviation.
Q = lowest average quadrant activity on stress PET image, % of maximum activity in whole heart data set.
L = longitudinal base to apex perfusion gradient on stress PET image in SD units.
*p < 0.001 compared to normals and for ANOVA for differences between the three patient groups.

### Discussion

Coronary endothelial dysfunction refers to a wide spectrum of coronary vasomotor pathophysiology associated with preclinical and clinical CAD that may involve epicardial arteries or microvasculature, different vasoactive mediators, different stimuli, and different pathophysiologic or clinical manifestations. However, assessing coronary endothelial dysfunction and application of extensive research knowledge have not been clinically developed because of its complexity and lack of noninvasive approaches. A new concept in perfusion imaging is disclosed by demonstrating a close relation between resting perfusion heterogeneity outside normal limits with early or advanced coronary disease in a large number of patients with well-defined risk factors based on the association of endothelial dysfunction with microvascular dysfunction.

Endothelial dysfunction as a cause of coronary arterial and microvascular vasoconstriction is well documented in experimental studies and in humans by coronary arteriography or Doppler flow-velocity wires or catheters. Vascular mediators derived from coronary endothelium include prostacyclin, nitric oxide, thromboxane, endothelin, bradykinin, angiotensin, serotonin, substance P, C-type naturetic peptide ([CNP], an endothelium-derived hyperpolarizing factor), and others. The mechanisms may be inhibition of normal vasodilatory mediators such as nitric oxide or activation of vasoconstrictor mediators such as endothelin. The stimuli, mediators, and the vascular responses of epicardial coronary arteries and the coronary microvasculature are quite different, even divergent. For example, in the epicardial coronary arteries, acetylcholine-induced vasodilation is mediated by nitric oxide. However, in the coronary microcirculation, acetylcholine-induced arteriolar vasodilation and increased coronary flow are not mediated by nitric oxide. With epicardial artery endothelial dysfunction, acetylcholine causes arterial vasoconstriction while arteriolar vasodilation with increased flow remains intact as an example of divergent pathophysiologic behavior of the macrovasculature and microvasculature of the heart.

As a further example, endothelial nitric oxide production mediates epicardial coronary artery vasodilation during exercise but is not involved in arteriolar vasodilation and increased coronary flow during exercise unless there is a flow-limiting stenosis in which nitric oxide helps maintain perfusion during exercise. In opposition to these vasodilator mechanisms, endothelin is a powerful coronary arteriolar vasoconstrictor that is activated in coronary atherosclerosis in parallel with inhibition of nitric oxide production.

Thus, there is no single specific vasomotor abnormality, gold standard, diagnostic test, or even definition that identifies or defines coronary endothelial dysfunction. The present data indicate that resting myocardial perfusion heterogeneity is one manifestation of this wide spectrum of coronary vascular behavior that is a powerful independent predictor of preclinical CAD, more than standard risk factors. In view of the different, sometimes divergent, arterial and arteriolar behaviors in response to the wide variety of vasoactive mediators, resting perfusion heterogeneity would not necessarily be expected to parallel the effects of intracoronary acetylcholine or cold pressor testing, just as the arteriolar response to acetylcholine with increased blood flow does not parallel its vasoconstrictive effect on epicardial coronary arteries in CAD.

It should be noted that the limited resolution of PET cannot resolve the small regions of heterogeneous perfusion previously described in experimental animals or the subendocardial underperfusion that is an effect of flow-limiting stenosis. The heterogeneity that is visually apparent and objectively quantified in this example involves regions of myocardium greater than the 1-cm³ scanner resolution, consistent with the arterial distribution of coronary arteries and their secondary or tertiary branches demonstrated to have heterogeneous endothelial function by coronary arteriography and intracoronary Doppler flow-velocity measurements. Therefore, the heterogeneity that is observed by PET perfusion imaging is separate and unrelated to the dispersion of perfusion in small 1-mm myocardial samples for microsphere measurements of perfusion reported for experimental animals.

The heterogeneous resting perfusion was quantified separately from, independently of, and around significant regional perfusion defects caused by flow-limiting stenosis and, therefore, does not involve subendocardial hypoperfusion due to reduced perfusion pressure or reduced early diastolic subendocardial filling caused by flow-limiting stenosis.

The limits of heterogeneity were determined from 50 healthy control subjects imaged on the same scanner and software as the patients so that the technical limitations of PET or any potential effects of microscopic dispersion apply equally to both sets of subjects with the significant differences reported here. The application of homogeneity analysis to PET perfusion images requires careful attention to the technical details of cardiac PET with ⁸²Rb that are different than those required for cancer PET, including the necessity of lower spatial resolution in favor of a high count density, 2D imaging to reduce scattered radiation, highcount, low-noise, filtered backprojection reconstruction, and compulsive correction of emission-transmission image coregistration.

Coronary arteriography was not performed or used as a comparative gold standard in all of these patients. The percentage diameter stenosis as a measure of the severity of CAD is notoriously inadequate because of diffuse disease. The base-to-apex longitudinal perfusion gradient by PET perfusion imaging identifies early diffuse CAD better than regional stress-induced perfusion defects of flow-limiting stenosis. Because coronary atherosclerosis is a continuous spectrum from early mild stages to severe stenosis, the conventional categorization of arteriograms or perfusion images into “normal” or “abnormal” for determination of sensitivity or specificity is artificial and incorrect, particularly when defined as outside 2 SD of normal. The present multivariate regression analysis using continuous quantitative variables confirms the continuous spectrum of these endpoints. Accordingly, for added certainty, logistic regression analysis was performed using as thresholds of the endpoints a cutoff of <1 SD as “not normal,” that is, a greater probability of being “abnormal” than “normal” to include the great extent of mild preclinical CAD with potential for plaque rupture and coronary events. Although resting perfusion heterogeneity or its improvement is associated with “not normal” stress perfusion PET scans, some patients with resting perfusion heterogeneity had normal stress perfusion PET scans. By association with otherwise comparable patients with stress-induced perfusion changes, the findings suggest that such patients with heterogeneity or its improvement without stress-induced defects are at risk for vascular disease.

Accordingly, it is concluded from this example that patchy diffuse heterogeneity of resting myocardial perfusion by noninvasive PET quantified objectively by automated software using Markovian mathematic analysis is a powerful independent predictor of even mild stress-induced perfusion defects or base-to-apex longitudinal perfusion gradients of diffuse CAD and is more predictive than standard risk factors, consistent with coronary microvascular dysfunction-associated early or advanced CAD for potential preventive treatment.

### Example 2

### Protocol for Demonstrating Improvement of Myocardial Perfusion Heterogeneity with Administration of a Selective ET_{A}-Receptor Antagonist

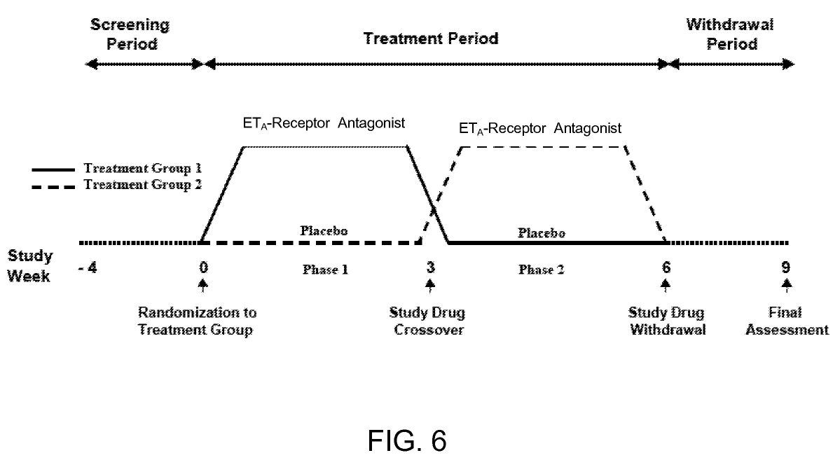

FIG. 6 is a schematic flow diagram illustrating the drug and placebo administration protocol for a study to demonstrate that myocardial perfusion heterogeneity, quantified by Markovian Homogeneity analysis of cardiac PET perfusion images, will improve after treatment with a selective endothelin receptor A antagonist (ET_{A}-receptor antagonist), compared to treatment with placebo.

The visually apparent heterogeneity of resting myocardial perfusion and/or its improvement after dipyridamole or adenosine stress on high quality, non-invasive PET images is proposed as one manifestation of coronary arteriolar endothelial dysfunction. Positron emission tomography (PET) is necessary for imaging this pattern of heterogeneous perfusion without the attenuation artifacts and poor depth dependent resolution of standard single photon emission tomography (SPECT). The application of Markovian Homogeneity Analysis to myocardial PET perfusion images provides the first automated, objective methodology for precisely quantifying the visual heterogeneous perfusion pattern, also reported in the literature by Johnson and Gould, J Nucl Med 46: 1427-1437, 2005, the disclosure of which is hereby incorporated herein by reference. It is proposed that coronary endothelial dysfunction is associated with pre-clinical coronary atherosclerosis preceding the earliest stages of coronary artery disease in association with increased risk of coronary events and subsequent clinically manifest coronary artery disease many years later, thereby providing a basis for intense, lifelong, pharmacologic and lifestyle preventive treatment.

It is further proposed that myocardial perfusion heterogeneity, quantified by Markovian Homogeneity analysis of cardiac PET perfusion images, will improve in a quantitative manner after treatment with a selective ET_{A}-receptor antagonist, such as Sitaxsentan™ (Barst et al. Am J Respir Crit Carer Med 169:441-7, 2004), Darusentan™, BQ123 (Kolettis et al., Interv Card Electrophysiol 8:173-9, 2003), BMS1822874 (Bristol Meyers Squibb), PD156707TTA101 (Pfizer), 34-sulfatobastadin (Novartis) (Davenport and Battistini, Clin Sci (Lond.) 103 Suppl 48:1S-3S, 2002) and BSF302146 ([+]-[S]-2-[4,6-dimethyl-pyridimin-2-yloxy]-3,3-diphenyl-butanoic acid) Wan et al. Thorac Cardiovasc Surg 127:1317-22, 2004). A “selective” antagonist is active for blocking the ET_{A} receptor and not the ET_{B} receptor. For instance, the antagonist Darusentan may be administered 100 mg per day for 3 weeks and compared to baseline and post-treatment PET scans in clinically stable subjects with coronary atherosclerosis and/or risk factors).

A 9-week randomized, double-blind, crossover, investigator-initiated, single-center study will reveal the beneficial effect of a representative ET_{A}-receptor antagonist (“drug”), e.g., Darusentan™, administered 100 mg once daily on myocardial perfusion heterogeneity in subjects with documented CAD, as measured by cardiac PET imaging. Screening assessments and evaluations may be conducted over a period of not more than 4 weeks. Following a baseline PET scan (PET 1) subjects will be randomized to one of two treatment groups (Group 1 or Group 2), and receive blinded treatment for a total of 6 weeks. The 6-week treatment period will have two phases, Phase 1 and Phase 2. Group 1 will receive the drug 100 mg for 3 weeks during Phase 1, then placebo for 3 weeks during Phase 2. Group 2 will receive placebo for 3 weeks during Phase 1, then drug 100 mg for 3 weeks during Phase 2. Following 6 weeks of treatment with blinded study drug, subjects in both treatment groups will have study drug withdrawn for an additional 3 weeks. Maximum drug exposure will be 3 weeks, and maximum placebo exposure will be 3 weeks. Adjustments to the number or dosage of concomitant medications required for study entry will not be permitted.

Efficacy will be assessed through cardiac PET imaging. In total, 4 PET scans will be administered: the first at the Randomization Visit (PET 1, Week 0); the second at the conclusion of Phase 1 (PET 2, Week 3); the third at the conclusion of Phase 2 (PET 3, Week 6) and the fourth at the conclusion of the withdrawal period (PET 4, Week 9).

Subjects will take their study drug (i.e., placebo or drug, respectively) with or without food once daily at approximately the same time in the morning throughout the course of the study. Subjects will also be instructed to take all concomitant medications consistently and at the same time each day throughout the study.

It is expected the Markovian Homogeneity Number, a value that quantitates myocardial perfusion heterogeneity will change during drug treatment. It is also expected that the size and severity of regional perfusion defects will change, and the base to apex longitudinal perfusion gradient will also change during drug treatment. It is expected that these changes will be observed at rest and following dipyridmole stress by PET perfusion imaging, and will also be measurable by automated software. A representative study design for demonstrating the efficacy of an endothelin receptor A antagonist in improving myocardial perfusion homogeneity is shown schematically in FIG. 1. A representative schedule of assessments of test subjects is shown in Table 6.

TABLE 6
Schedule of Assessments

| Study Week | | | | | |
|---|---|---|---|---|---|
| | 0¹ | 3 | | 9 | |
| −4 | Treatment | Treatment | 6 | Final | Premature |
| Screen | Phase 1 | Phase 2 | Withdrawal | Assessment | Discontinuation |

Assessments

| | | | | | | |
|---|---|---|---|---|---|---|
| Signed ICF/HIPAA | X | | | | | |
| Authorization | | | | | | |
| Review | X | X | | | | |
| Inclusion/Exclusion | | | | | | |
| criteria | | | | | | |
| Adverse Event | | X | X | X | X | X |
| Assessment | | | | | | |
| Vital Signs | X | X | X | X | X | |
| Physical | X | | | | X | X |
| Examination | | | | | | |
| PET scan² | | X | X | X | X | |

Laboratory Tests

| | | | |
|---|---|---|---|
| Chemistry | X | X | X |

Study Drug

| | | | | | |
|---|---|---|---|---|---|
| Randomization | X | | | | |
| Dispense study | X | X | | | |
| drug | | | | | |
| Collect unused | | X | X | X | X |
| study drug/Assess | | | | | |
| compliance | | | | | |

¹Randomization Visit. This visit occurs no more than 28 ± 3 days after the Screening Visit.
²The PET imaging procedure includes 12-lead ECG and blood pressure monitoring.

### PET Image Acquisition

PET scans are obtained as previously described with a Positron Corporation Scanner with resolution of 8 to 10 mm FWHM. Using a rotating rod source containing 4-5 mCi of Gallium 68, transmission images to correct for photon attenuation are obtained over 20 minutes containing 50 to 100 million counts. Emission images of 20 to 60 million counts are obtained over 5 to 6 minutes following 25 to 60 mCi of generator produced Rb-82. To allow for blood pool clearance, image acquisition is begun after a 60 second delay following onset of Rb-82 infusion. After completing resting Rb-82 images, dipyridamole (0.56 m/kg) is infused for 4 minutes. Four minutes after the dipyridamole infusion is completed, a second dose of the same amount of Rb-82 is injected, and imaging is repeated. For those patients developing significant angina, aminophylline 125 mg is given intravenously.

### Automated Quantitative Analysis of PET Images

Images are reconstructed using Filtered Back Projection with a Butterworth filter having a cut off of 0.4 and roll off of 10. Completely automated analysis of severity and size of PET abnormalities are carried out by previously described software based on the concept of coronary flow reserve. A three-dimensional restructuring algorithm generates true short and long axis views from PET transaxial cardiac images, perpendicular to and parallel to the long axis of the left ventricle. From image data acquired in two-dimensional tomographic mode in order to minimize scatter, circumferential profiles are used to reconstruct three dimensional (3-D) topographic views of the left ventricle showing relative regional activity distribution divided into lateral, inferior, septal and anterior quadrant views of the 3-D topographic display corresponding to the coronary arteries.

Mean activity in each quadrant is normalized to the maximum 2% of pixels in the whole heart data set. Regions of each quadrant are identified having values outside 97.5% confidence intervals (CI) or 2.5 standard deviations (SD) outside normal values of 50 healthy control subjects with no risk factors by complete medical history. Percent of circumferential profile units outside 97.5% CI are calculated automatically. Every rest-dipyridamole myocardial PET is checked and corrected for any misregistration of attenuation and emission images commonly seen in association with artifactual defects.

### PET Endpoints

The PET endpoints are three separate, independent measures of perfusion abnormalities at rest and after dipyridamole stress, measured objectively by automated software as outside 2.5 standard deviations of 50 normal healthy controls for the following endpoints (1) size and severity of regional perfusion defects, (2) the base to apex longitudinal perfusion gradient due to diffuse CAD before localized flow limiting stenosis, and (3) Markovian Homogeneity Analysis for the patchy heterogeneous perfusion pattern associated with endothelial dysfunction. Each of these categories of end points is detailed below.

### Size and Severity of Regional Perfusion Defects

(i) The endpoint lowest quadrant average is the average number of normalized counts for the quadrant having the lowest average activity, where there is an anterior, septal, lateral and inferior quadrant surrounding a central apex area that is analyzed separately. The mean value for any given quadrant that is the lowest, or minimum, contains the perfusion defect. This lowest quadrant average determined for the PET image at rest and after dipyridamole stress quantifies the severity of the perfusion abnormality. For example, a value of 65% indicates that the mean count value for the quadrant with the lowest counts, and therefore containing the perfusion defect, is 65% of the normal maximum of 100%.

(ii) The endpoint, percent outside 2.5 standard deviations (SD), is the size of the perfusion defect determined as the percent of the cardiac image outside of 2.5 SD of normals for the PET image at rest and after dipyridamole stress. Since 2.5 SD include 97.6% of the normal distribution, there is only a 2.4% chance that normal values outside of 2.5 SD would be observed.

(iii) The end point, percent with activity<0.6, is a measure of combined size and severity determined as percent of myocardium with activity of less than 60% of maximum activity (100%) on the PET image. This measurement gives the size of the defect characterized by the severity threshold of less than 60% of normal maximum of 100%. It therefore reflects both the combined intensity and size of defect. A value<0.6 or <60% of maximum on the PET image is approximately 3 SD below the normal mean of maximum activity. Since 3 standard deviations contain 99.7% of the normal distribution, there is less than a 0.3% chance that normal values would be observed below 60% of maximum activity.

(iv) The endpoint, % of the cardiac image in the top 80% to 100% of relative activity is a measure of the highest perfusion in the heart that may improve more than areas supplied by flow limiting stenosis in association with improved endothelial function. It therefore reflects the changes in the best perfused segments of the heart rather than the worst perfusion defects.

### Base to Apex Longitudinal Perfusion Gradient

Relative activity of the cardiac image is graphed on the vertical axis with slice number on the horizontal axis for each tomographic slice from base to apex for each quadrant and for the whole heart. These graphs of the base to apex activity distribution are best fit to a third degree polynomial equation and the first derivative or spatial slope determined thereby providing a single number that quantifies the base to apex change in activity. The slopes are typically negative thereby indicating diminishing activity from base to apex with narrow normal limits for 50 normal healthy subjects for comparison to patients. The slope of the base to apex longitudinal gradient outside normal limits is a marker of diffuse coronary atherosclerosis before flow limiting stenosis develops.

### Application of Homogeneity Analysis to PET Perfusion Images

Markovian homogeneity analysis is carried out as described in Example 1. Two topographic maps are produced for each patient, representing the rest and stress scans. Each topographic map consists of 21 slices along the long-axis of the left ventricle. Every long-axis slice contains 64 radial pixel units, representing equal angles around a circle (360 degrees divided over 64 pixel units equals just under 6 degrees per pixel unit). The four quadrant views (lateral, inferior, septal, and anterior) contain 16 radial pixel units each. Radial pixel units originally have integer values between 0 and 1000, where 0 represents no activity or no relative activity and 1000 represents maximum activity or maximum relative perfusion.

Before applying Equation 1 to a topographic map, three modifications were made as follows (i) the basal 4 slices are discarded to avoid count variability in the membranous septum and the apical two slices are discarded to minimize partial volume effects and variability in locating the last apical slice (ii) pixels with intensity values below 500 are reset to 500, and pixels with intensity values above 850 are reset to 850 in order to eliminate any effect on H of very low activity levels of infarcted myocardium as well as eliminate any effect of the highest activity levels of normal myocardium. In effect, these limits confine the homogeneity analysis to relative activity values ranging from 50% to 85% of maximum on each PET image, thereby excluding extreme values as from severe defects or hot spots that would bias the value of H intended to reflect more subtle differences amongst pixel units (iii) these modified intensity values, 500 to 850 inclusive, are scaled into an integer range of 35 levels, so that each new intensity level represents 1% of the original intensity range from 0 to 1000.

More intensity levels would tend to decrease lower-order terms like Pd(0) and Pd(1) and increase higher-order terms like Pd(2) and Pd(3) that both would decrease the Homogeneity Index. The choice of 35 intensity levels for the original range of values of 50% to 85%, as 1% differences in intensity, serves to mathematically restrict the analysis on myocardium that is neither infarcted, severely ischemic, or maximally perfused. Consequently, the Homogeneity Index is not greatly influenced by severe perfusion defects. The degree of small scale heterogeneous “patchyness” is objectively quantified on resting and stress images as well as the rest-to-stress change independently of, separately from, or around any more severe discrete regional perfusion defects due to myocardial scar, reduced coronary flow reserve of flow limiting stenosis or the base to apex longitudinal perfusion gradient due to diffuse disease.

### Visual Analysis of PET Studies

Baseline and final, rest and dipyridamole images in three dimensional topographic format are displayed together for direct side by side comparison by readers blinded to identity, clinical information and treatment drug. Hard color copy print outs are saved in each patients file. Subjects will be instructed to fast for 4 hours and abstain from caffeine including decaffeinated beverages, theophylline and cigarettes for 24 hours prior to study. Decaffeinated beverages should also be avoided as there is residual caffeine in people who metabolize it slowly that may reduce the response to dipyridamole. An intravenous catheter is inserted. Patients are monitored by 12-lead ECG and automatic blood pressure monitoring.

### Statistical Methods

The primary endpoint is the Markovian Homogeneity number, ranging from 0 to 1 for each subject at each time point. The values will be summarized with n, mean, median, standard deviation, minimum and maximum for each treatment group at each PET scan: baseline, 3 weeks, 6 weeks and 9 weeks. Secondary endpoints, including size and severity of defect and baseline to apex longitudinal gradient, will be similarly summarized. Assuming normality in the change of Homogeneity numbers and an observed standard deviation of 0.15, a two-sided 95% confidence interval for the true standard deviation, useful for planning future studies, will be 0.11 to 0.22.

### Example 3

### Demonstration of Endothelin-Induced Myocardial Perfusion Defects

In diagnostic myocardial perfusion imaging, the resting perfusion image serves as a baseline for comparison to the exercise or pharmacological stress image where a new or worsening stress-induced perfusion abnormality indicates flow-limiting stenosis. This paradigm of rest-stress perfusion imaging is based on the concept of coronary flow reserve and perfusion imaging during pharmacological stress for assessing coronary artery stenosis. In the absence of attenuation artifacts as with positron emission tomography (PET), a persisting fixed perfusion defect is clinically interpreted as myocardial scar or hibernating myocardium due to flow-limiting stenosis.

However, resting myocardial perfusion defects that improve or disappear during dipyridamole stress in the absence of myocardial scar or flow-limiting stenosis have also been described. In Example 1, it was demonstrated that myocardial perfusion heterogeneity at rest and/or after dipyridamole stress quantified by Markovian homogeneity analysis is closely associated with early nonobstructive coronary artery disease (CAD). However, the mechanisms underlying these resting perfusion abnormalities in the absence of myocardial scar or flow-limiting stenosis have not been identified. Endothelin-1 (ET-1) is the strongest known arteriolar vasoconstrictor peptide and contributes to resting coronary vasomotor tone and coronary spasm due to localized paracrine effects more than blood concentrations. Vasoconstriction due to ET-1 and associated reduction in coronary flow is at least twice as potent as the flow reduction caused by inhibiting nitric oxide synthesis with N^{G}-monomethyl-L-arginine (L-NMMA) that does not change coronary phasic flow or maximum flow capacity despite reduction in coronary artery diameter. There is corresponding uneven heterogeneous distribution of myocardial perfusion among different segments of the same coronary artery or different coronary arteries after acetylcholine-induced coronary vasoconstriction. ET-1 is mitogenic for smooth muscle cells and is associated with atherosclerosis progression. Elevated plasma levels of ET-1 are found in patients with chest pain and normal coronary arteries, in diabetes, obesity, hypertension, CAD, acute coronary syndromes, congestive heart failure, and slow coronary flow transit time at arteriography and after coronary stenting, all associated with coronary endothelial dysfunction. Adenosine is a powerful coronary vasodilator used for myocardial perfusion imaging to identify flow-limiting coronary artery stenosis. It is predominantly a direct smooth muscle vasodilator. Therefore, it was tested in an animal model the hypothesis that intracoronary ET-1 may cause myocardial perfusion abnormalities by PET at resting conditions that may persist or only partially improve after intravenous adenosine stress in the absence of myocardial scar and flow-limiting stenosis. The tests are also described in the publication of Loghin C, Sdringola S, Gould K L, “Does coronary vasodilation after adenosine override endothelin-1-induced coronary vasoconstriction?” Am J Physiol Heart Circ Physiol 292:496-502, 2007.

Experimental Preparation. The experimental protocol was approved by the Animal Welfare Committee of the University of Texas Health Sciences Center at Houston. After an overnight fast, healthy adult hound dogs (n=14) of both sexes, 21-35 kg, were anesthetized with 30 mg/kg pentobarbital sodium (Nembutal) and underwent endotracheal intubation and mechanical ventilation with adequate anesthesia maintained by small supplemental doses during the experiment. Arterial blood gases were maintained within physiological range by adjusting the mechanical ventilator with supplemental oxygen, core body temperature was maintained at 37° C. with a homeothermic blanket, and stable body position was maintained throughout the duration of the imaging protocol by using a specially designed portable cradle for both coronary arteriography and PET imaging without moving the dog strapped to the portable cradle.

Arterial access was obtained via the right femoral artery by standard Seldinger technique with an arterial micropuncture kit (Cook, Bloomington, Ind.) with a 6-F arterial sheath. All animals received 100 IU/kg of initial heparin bolus, with 50 IU/kg every hour for the duration of the experiment. A 6-F standard JL3 VistaBrite coronary guide catheter (Cordis-Cardiology, Miami Lakes, Fla.) was positioned under fluoroscopic guidance into the left main coronary artery ostium.

After a stable position for the guide catheter was obtained, a 0.014-in.-diameter Hi-Torque Whisper guide wire (Guidant, Indianapolis, Ind.) was placed in the left circumflex coronary artery (LCx). Over the coronary wire, a 2.3-F, 150-cm-long, 0.042-in.-minimal diameter Rapidtransit infusion catheter (Cordis-Cardiology) was positioned in the LCx, with the tip in the midsegment of the LCx artery proximal to the takeoff of the first large obtuse marginal branch. The guide wire was removed, the guide catheter was withdrawn into the aortic root, and the small catheter was left in place in the mid-LCx.

Experimental Protocol. Contrast angiograms were obtained to document the LCx as the dominant coronary artery and the small catheter position in the mid-LCx. With contrast solution diluted 50% with normal saline, different injection rates were tested to determine the rate at which back flow occurred into the proximal segment of the coronary artery. No back flow was observed at infusion rates of <5 ml/min. Dogs were then moved into the PET scanner without changing position on the special cradle, and a repeat subselective angiogram was performed under fluoroscopy on the PET imaging table to ensure that the small intracoronary infusion catheter positioned in the mid-LCx had not changed during transportation. PET imaging was carried out with the University of Texas-designed Posicam BGO multislice tomograph HZL/mPower (Positron, Houston) as previously described (e.g., Halcox et al., Circulation 106:653-658, 2002), with a reconstructed resolution of 10-mm full-width half-maximum. Images were acquired in two-dimensional mode with extended septa to minimize scattered counts with random coincidences corrected from the singles count rate. Images were reconstructed with filtered back projection, with a Butterworth filter order of 5 and 0.04 cycles/mm corresponding to a cutoff of 0.16 for the input pixel dimensions of 2×2×2.6 mm, displayed with image dimensions of 256×256.

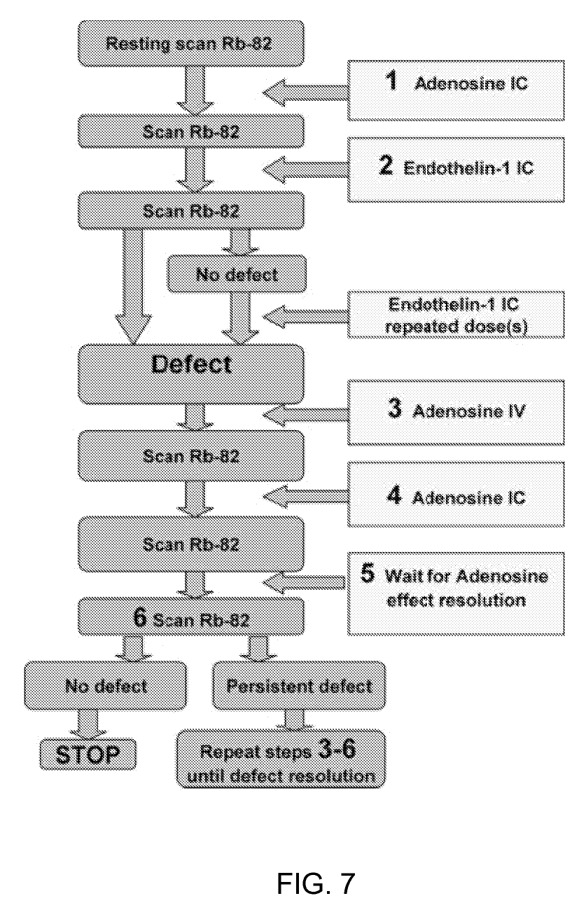

On the basis of a 5-min positioning transmission scan, dogs were precisely positioned in the PET scanner and laser guides aligned to external body markers were used to check correct position for every image acquisition. With a rotating rod source containing 4-5 mCi of ⁶⁸Ge, transmission images to correct for photon attenuation contained about 70-90 million counts. Emission images obtained after intravenous injection of 935-1,110 MBq (25-30 mCi) of generator-produced rubidium-82 (⁸²Rb) contained about 20-30 million counts. The protocol used the following imaging sequence as illustrated in FIG. 7.

Step 1. Immediately after completion of a resting perfusion image with intravenous ⁸²Rb, 3 mg/ml adenosine (Fujisawa Healthcare, Deerfield, Ill.) was infused subselectively into the LCx for 4 min at 2 ml/min. Two minutes before the end of infusion, a second dose of ⁸²Rb was injected intravenously and images were obtained to confirm position of the coronary infusion catheter in the mid-LCx, to document the distribution and size of the myocardial LCx territory perfused distal to the catheter, and to quantify the relative increase in activity over baseline induced by intracoronary adenosine.

Step 2. ET-1 (Sigma-Aldrich, St. Louis, Mo.) was then infused into the LCx via the subselective intracoronary infusion catheter at an initial dose of 1.5-3.5 ng·kg⁻¹·min⁻¹ for 10 min in nine dogs. Myocardial perfusion imaging was repeated with intravenous ⁸²Rb. In six dogs, it was necessary to administer repeated smaller doses of ET-1 in order to obtain a perfusion defect on PET images. The solution of ET-1 was prepared to provide an infusion rate of 2-2.5 ml/min for any given dose rate in order to avoid backfilling demonstrated only at above two times this infusion rate at coronary arteriography. No tachyarrhythmia developed, and no animal died because of ET-1 administration.

Step 3. After the ET-1 image was acquired, adenosine was then given intravenously at a dose of 0.142 mg·kg⁻¹·min⁻¹ for 6 min. ⁸²Rb was administered intravenously 3 min before the end of adenosine infusion and imaging was repeated.

Step 4. Adenosine was then given as an intracoronary injection via the coronary infusion catheter at the same dose and rate as the initial intracoronary adenosine injection. ⁸²Rb was administered 2 min before the end of the intracoronary adenosine infusion, and imaging was repeated.

Step 5. After effects of the last adenosine injection had abated, another emission scan was gain performed. If a perfusion defect persisted in the LCx territory, the sequence of intravenous and intracoronary adenosine was repeated. The images showing a persistent defect were used as a reference for analysis of subsequent scans after additional adenosine injections. This follow-up imaging provided up to three serial protocol sequences of ET-1-induced perfusion defects, intravenous adenosine, and intracoronary adenosine, resulting in a total of 23 protocol sequences for the 9 dogs in this protocol.

Step 6. In five additional dogs, instead of infusion of ET-1 as in step 2 above, the nitric oxide synthesis inhibitor L-NMMA (Paragon Biochemical) was infused into the LCx via the coronary infusion catheter at doses ranging between 100 and 400 μg·kg⁻¹·min⁻¹ for 10 min at the same flow rate to avoid backfilling. Perfusion images were again obtained after administration of intravenous ⁸²Rb.

Step 7. A final emission scan was acquired at the end of each experiment to document persistence or resolution of the perfusion defect over the total time of the experiment. Normal saline was infused through the coronary infusion catheter at a rate of 2 ml/min between all drug administrations to ensure catheter patency. At the completion of the protocol, dogs were euthanized by an injection of potassium chloride (50 meq iv).

In an initial pilot phase, dose-finding experiments were conducted on three additional separate dogs to identify the intracoronary ET-1 dose that was necessary to induce a resting perfusion defect, to determine the time required for a perfusion defect to develop after intracoronary ET-1 infusion, and to measure duration of the perfusion defect. A perfusion defect was observed as early as the end of the initial 10-min intracoronary ET-1 infusion and lasted in several instances up to 5 h. Consequently, the total cumulative ET-1 weight-based dose incorporating the number and duration of administrations was calculated. If several ET-1 injections were administered, all were given within an interval of 90 min.

Quantifying Relative Changes in PET Images. Activity in each cardiac image data set was normalized to the maximum 2% of pixels in the whole heart data set in order to obtain a relative normalized scale as well as the original scale of absolute counts. The purpose of this first normalization of all activity in the heart to its maximum counts was to enable combination of data from all studies in all animals with the least measurement variability so that small relative regional changes below resting could be reliably measured. Relative changes on PET perfusion images were used for four reasons. 1) Relative perfusion defects, i.e., relative coronary flow reserve, are independent of heart rate and perfusion pressure, whereas absolute flow and absolute coronary flow reserve are highly dependent on heart rate and blood pressure changes. 2) The relative perfusion defects after intravenous injection of radiotracer are comparable to relative defects of clinical imaging and need to be studied as relative defects if they are to be relevant to clinical PET perfusion imaging as now most commonly performed. 3) The reproducibility of quantifying relative defects is very good, with one SD of repeated measurements of relative defects being 0.5% in this example. For comparison, in a comprehensive review of 23 publications, the SD of absolute perfusion expressed as a percentage of mean flow is 24% (SD 12) for absolute resting perfusion and 29% (SD 12) for stress absolute perfusion, reflecting much greater variability than the SD of 0.5% for relative defects in this study. 4) Since the scientific question addresses only small relative changes below resting perfusion, this approach was designed to provide precise measurements of small relative regional perfusion defects in resting perfusion images without needing to measure relative changes above resting levels, coronary flow reserve, or absolute perfusion having substantially greater methodological variability than the relative changes expected here.

Small relative regional changes in the LCx distribution were determined on whole heart-normalized images as follows. A pixel size of 2×2 mm in the tomograpic plane best showing the perfusion defect in the distribution of the LCx after intracoronary ET-1 was selected that also had the highest counts on the resting baseline image, and x, y, and z coordinates were recorded. For each location with the highest baseline counts in the LCx distribution on the resting baseline image, the pixel value normalized to the whole heart was recorded for all images—at baseline and for each intervention.

To obtain the primary end point data, the normalized LCx pixel value on the image after an intervention was divided by the normalized pixel value on the resting baseline image before the intervention at the same LCx location; this ratio is multiplied by 100 to express the LCx pixel value after the intervention as a percentage of the resting baseline pixel value before the intervention. The baseline pixel coordinates were used to locate and record the pixel values on all subsequent images for determining the changes after subsequent interventions with ET-1 and adenosine or L-NMMA.

The method of quantifying these relative changes in radionuclide uptake in the LCx distribution has some notable specific characteristics. Small changes in relative normalized radionuclide uptake in the LCx distribution can be precisely determined independently of activity in the left anterior descending coronary artery LAD region and without the biological and methodological variability of absolute perfusion measurements. Furthermore, relative perfusion defects are relatively independent of heart rate and blood pressure changes compared with absolute flow measurements. Moreover, the marked increased perfusion in the LCx distribution after intracoronary adenosine is normalized out for the following reason. At baseline, perfusion is uniform and pixel values normalized to the maximum activity are uniformly 100% throughout the heart, including the LCx area. After administration of intracoronary adenosine into the LCx and intravenous ⁸²Rb, the activity in the LCx area is the maximum activity in the heart where the pixel values are therefore also 100%. Thus the LCx pixel value normalized to maximum activity at baseline is 100%, and the same pixel value normalized to maximum activity after intracoronary adenosine is also 100%. Consequently, the ratio of the normalized pixel values after adenosine to baseline is also 1.0 or 100% and does not reflect an increase due to intracoronary adenosine.

Intracoronary adenosine with intravenous ⁸²Rb after the baseline image was used solely for confirming the position of the small subselective catheter in the LCx, not for obtaining end point data. For testing the present hypothesis on resting perfusion defects, assessing coronary flow reserve or the increases in perfusion over baseline after adenosine was not important since testing of the hypothesis required precisely measuring relative small decreases in perfusion below resting levels. If the measurement technique incorporated a wide range from zero to four times baseline, then the measurements of small decreases below resting values would have less precision and greater variability, like the high- versus low-range options on a voltmeter. Since one of the scientific questions in this example addresses only the small relative changes below resting perfusion, this approach was designed to provide precise measurements of small relative regional decreases in resting perfusion images without needing to measure relative changes above resting levels, coronary flow reserve, or absolute perfusion having substantially greater methodological variability than the relative changes expected.

Activity in the LCx distribution to the LAD distribution was intentionally not normalized because 1) intravenous adenosine would increase the perfusion in the LAD distribution that would then change the LCx-to-LAD ratio regardless of small positive or negative changes in the LCx distribution that were of specific interest in this protocol and 2) referencing the LCx activity to the LAD activity after intravenous adenosine would in effect measure the relative coronary flow reserve of the LCx compared with the LAD. However, as explained above, assessing relative coronary flow reserve would not address the hypothesis about relative resting perfusion defects. Based on alignment of external markers, superimposition of the cardiac images, and the x, y, and z coordinates, there was no misregistration among baseline resting images and subsequent scans obtained after ET-1 and adenosine injections. Repeated readings of the entire data set showed a measurement variability of <0.5% relative uptake compared with the reported 24-29% variability in absolute flow measurements. In this example, there was no misregistration of attenuation and emission scans as has been previously reported for clinical studies.

Changes for each intervention study were quantified as percentage of the resting baseline pixel value before the intervention in the same LCx pixel as defined above. Therefore, for reporting the changes from resting baseline, the pixels selected on the baseline image had a value of 100%, with the value of the same LCx pixel after ET-1 expressed as some percentage below 100%.

Statistical Analysis. All statistical analyses were carried out with SPSS version 11.5 (SPSS, Chicago, Ill.). Data are reported as means (SD). Differences among the means of continuous variables were analyzed with an independent or paired two-tailed t-test. Levene's test for equality of variances was used to validate the t-test results. Analysis of variance was carried out for significance of variance, with Games-Howell post hoc test for unequal variances. Linear regression analysis was used to evaluate whether ET-1 dose predicted the quantitative response to adenosine. Spearman's nonparametric correlation test was used for correlating ET-1 dose and the subsequent response to intravenous adenosine. A two-tailed P value of <0.05 was considered statistically significant.

### Results

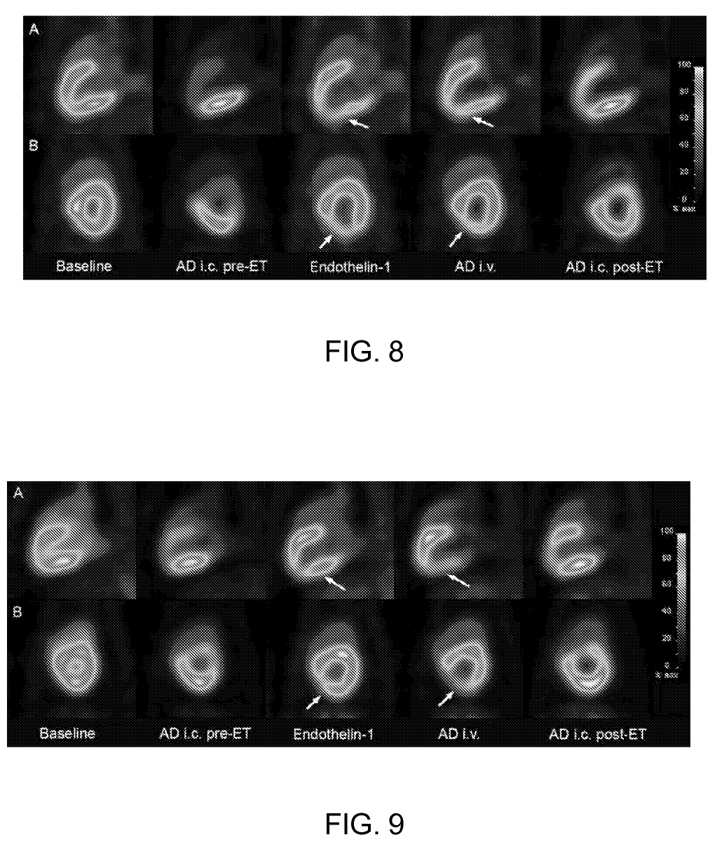

High-quality images were obtained. For each rest perfusion image, the dose of ⁸²Rb infused intravenously averaged 24.7 mCi (SD 0.8), the total number of counts for the baseline rest image data set averaged 23.1 million counts (SD 2.7 million), and the heart-to-lung ratio averaged 13.6 to 1 with SD of 4.6. The adenosine images were comparable with 24.7 mCi (SD 1.2) ⁸²Rb injected, 23.8 million counts (SD 2.8 million), and heart-to-lung ratio of 13.6 to 1 with SD of 4.6. The total counts for each image data set for the small chest and heart size of the dogs compared with humans produced very good images with high heart-to-lung ratios. FIG. 8 illustrates the series of PET perfusion images on this protocol: at resting control baseline, after intracoronary adenosine to confirm the location of the infusion catheter in the LCx, after intracoronary ET-1 infusion, after intravenous adenosine, after intracoronary adenosine used for quantifying the improvement in the ET-1-induced resting perfusion defects and after intravenous adenosine. Depending on the dose of intracoronary ET-1, the perfusion defect induced by ET-1 did not always improve after intravenous adenosine, as illustrated in FIG. 9, showing PET perfusion images in the same sequence. However, in all experiments, the ET-1-induced resting perfusion defects normalized after intracoronary adenosine.

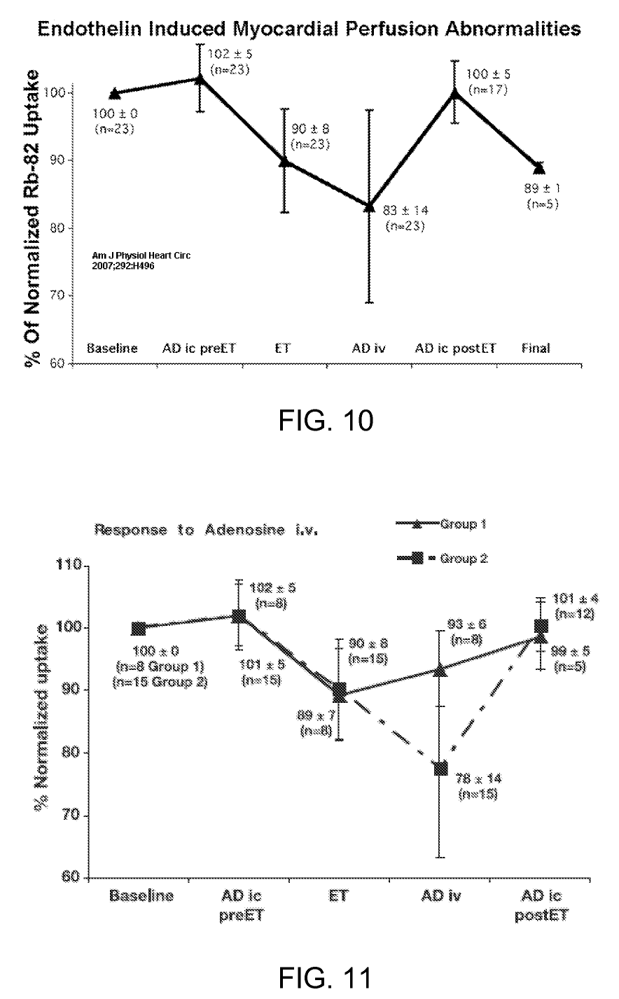

FIG. 10 shows the value of the LCx pixel as percentage of the baseline pixel values for all 23 complete protocol sequences obtained with PET perfusion imaging at resting control baseline, after initial intracoronary adenosine, after intracoronary ET-1, after intravenous adenosine, and after intracoronary adenosine and the final image with a persisting ET-1-induced defect after the adenosine effects had worn off. Intravenous adenosine at doses comparable to those used in clinical practice only partially counteracted the vasoconstrictor effect of ET-1 as opposed to intracoronary adenosine that induced vasodilation to a similar extent before and after intracoronary ET-1.

To analyze the differing effects of ET-1 on resting perfusion, the 23 complete protocol sequences were categorized into two groups based on the response to intravenous adenosine (Table 7). In group 1 (n=8), the ET-1-induced perfusion defect improved after intravenous adenosine as in FIG. 8. In group 2 (n=15), the ET-1-induced perfusion defect was visually not improved or relatively worse after intravenous adenosine, as shown in FIG. 9. Table 7 shows the differences in the severity of the perfusion defects related to the cumulative dose of ET-1 and the response to intravenous adenosine. There was no significant difference between the mean body weights in the two groups. The weight-based ET-1 infusion rate was not significantly different between the two groups [1.9 (SD 0.7) vs. 2.3 (SD 0.9) ng·kg⁻¹·min⁻¹; P=0.275], but this dose rate does not indicate the total cumulative dose of ET-1 given, which was significantly different between the two groups [44.3 (SD 13.9) vs. 67.5 (SD 28.9) ng; P=0.017].

TABLE 7
Response to Adenosine i.v. - Group Characteristics

| | Group 1 | Group 2 | |
|---|---|---|---|
| | (n = 8) | (n = 15) | p |
| Weight (kg) | 27.0 ± 4.4 | 29.2 ± 3.3 | NS |
| ET dose (ng/kg/min) | 1.9 ± 0.7 | 2.3 ± 0.9 | NS |
| ET total dose^{§} (ng) | 44.3 ± 13.9 | 67.5 ± 28.9 | 0.017 |
| PET | | | |
| PARAMETERS | | | |
| Baseline* | 91.2 ± 3.9 | 92.0 ± 5.2 | NS |
| AD i.c. pre-ET | 102.0 ± 5.5 | 101.3 ± 5.2 | NS |
| ET | 89.3 ± 7.3 | 90.2 ± 8.1 | NS |
| ΔET − Baseline | −12.7 ± 6.7 | −11.1 ± 11.9 | NS |
| AD i.v. | 93.4 ± 6.0 | 77.7 ± 14.5 | 0.008 |
| ΔAD i.v. − ET | 4.1 ± 2.0 | −12.5 ± 9.2 | <0.001 |
| AD i.c. post-ET | 98.7 ± 5.5 | 100.5 ± 4.3 | NS |
| ΔAD i.c − ET | 8.7 ± 4.3 | 10.5 ± 8.5 | NS |

*Expresses uptake as % of maximum Rb-82 uptake of the whole heart data set; all other PET data are expressed as % of peak activity in the LCx area normalized to activity of the same area on the baseline scan.
^{§}ET total dose represents total cumulative dose of intracoronary endothelin−1 calculated based on subject weight and total duration of administration.
AD = adenosine;
ET = endothelin−1;
pre-ET = injection prior to ET administration;
post-ET = injection post ET administration;
i.v. = intravenous;
i.c. = intracoronary;
Δ = difference.
All data expressed as mean ± 1SD;
NS = non-significant.

There was no significant difference between resting control baseline percent uptake relative to maximum whole heart activity of the two groups [91.2% (SD 3.9) vs. 92.0% (SD 5.2); P=0.675]. The response to intracoronary adenosine expressed as percentage of baseline was similar between groups 1 and 2 [102% (SD 5.5) vs. 101.3% (SD 5.2); P=0.98]. After initial ET-1 infusion(s) sufficient to produce a visible resting perfusion defect, there was no significant quantitative difference between the normalized percent uptake in the LCx territory in both groups [89.3% (SD 7.3) vs. 90.2% (SD 8.1); P=0.789], reflecting a similar severity of the ET-induced defect in both groups [−12.7% (SD 6.7) vs. −11.1% (SD 11.9); P=0.72].

After intravenous adenosine administration, group 1 showed visual improvement of the resting perfusion defect, quantified by a significantly different percentage of resting baseline pixel values compared with group 2 [93.4% (SD 6.0) vs. 77.7% (14.5); P=0.008]. The magnitude of the change, e.g., improvement for group 1 and worsening for group 2, was also significantly different between the two groups [+4.1% (SD 2.0) vs. −12.5% (SD 9.2); P<0.001]. The worsening of relative perfusion defects in the LCx distribution after intravenous adenosine in animals receiving high cumulative doses of ET-1 indicates that the normal surrounding or remote myocardium vasodilated after intravenous adenosine more than the LCx region that was vasoconstricted by high cumulative doses of ET-1 subselectively into the LCx coronary artery. In other dogs with less severe LCx vasoconstriction due to a lower cumulative ET-1 dose, intravenous adenosine vasodilates the LCx bed as much as the surrounding remote myocardium such that the relative defect is abolished. All ET-1-induced defects normalized after intracoronary adenosine, thereby proving functional vasoconstriction as the cause of the resting perfusion defects, not myocardial necrosis.

In contrast to the intravenous administration of adenosine, a subsequent intracoronary adenosine dose overcame the vasoconstrictor effect of ET-1 and induced similar complete defect resolution in both groups, with normalized percent uptake of 98.7% (SD 5.5) in group 1 vs. 100.5% (SD 4.3) in group 2 (P=0.473) after intracoronary adenosine. Defect resolution was also indicated by the difference between intracoronary adenosine and ET-1 images, expressed as percent change in LCx uptake of 8.7% (SD 4.3) for group 1 vs. 10.5% (SD 8.5) for group 2 (P=0.668). FIG. 11 shows the relative change in LCx uptake as percentage of the resting control baseline pixel values, over the protocol time line, for group 1 and group 2. The only significant difference is in the response to intravenous adenosine, whereas ET-1-induced resting defects and responses to intracoronary adenosine before and after ET-1 administration were similar between the two groups. The total cumulative dose of ET-1 correlated well with the quantitative change in the ET-1-induced defect after intravenous adenosine, expressed as the difference between adenosine and ET-1 percent change in LCx uptake (P=0.009; correlation coefficient −0.534). In a linear regression test, the total dose of ET-1 predicted the intravenous adenosine response intensity (P=0.055; confidence interval: −0.336 to −0.004).

In the five dogs receiving intracoronary L-NMMA, the perfusion defects were significantly less severe than after ET-1 [−2.48% (SD −4.11) for L-NMMA vs. −10.13% (SD 7.66) for ET-1; P=0.009], as expected since ET-1 is well recognized as the most powerful of endothelial vasoconstrictors. Consequently, ET-1 without L-NMMA was used for the remainder of the experiments.

Discussion. Intracoronary ET-1 causes visually apparent, quantitatively significant, localized, long-lasting resting myocardial perfusion defects that may persist or only partially improve after intravenous adenosine in doses comparable to those used in clinical diagnostic imaging in the absence of myocardial scar or flowlimiting stenosis. The degree of improvement after intravenous adenosine is inversely related to the total cumulative dose of ET-1. Since the canine model employed has normal coronary arteries without endothelial dysfunction, flow-limiting stenosis, or myocardial scar and the ET-1-induced perfusion defects normalized after intracoronary adenosine, this example demonstrates experimentally the concept that myocardial perfusion defects at rest and/or after intravenous adenosine stress may be due to a vasoactive mediator and not due to myocardial scar and flowlimiting stenosis.

The test results support the possibility that excess production of vasoconstrictors associated with severe endothelial dysfunction may partially explain the clinical finding of resting perfusion abnormalities or perfusion heterogeneity that partially improves or normalizes after adenosine or dipyridamole stress in the absence of myocardial scar or flow-limiting stenosis. The results of this example do not conflict with the initial demonstration of reduced coronary flow reserve and stress induced perfusion defects after pharmacological stress due to flow-limiting stenosis. Rather, it extends the understanding of myocardial perfusion imaging at rest and after adenosine stress.

It should be noted that, in this study, exogenous intracoronary ET-1 was used in an experimental model with normal coronary arteries in order to control precisely the experimental conditions for causing myocardial perfusion abnormalities. In addition, ET-1 is a powerful vasoconstrictor that is overexpressed in endothelial dysfunction associated with coronary atherosclerosis. Measuring plasma levels of ET-1 was not essential for several reasons. 1) The doses that were used have been shown to reflect pathophysiological concentrations of ET-1 in plasma. 2) Given the known abluminal path of endothelin secretion toward the myocardial interstitium rather than into the bloodstream, plasma endothelin concentrations do not reflect interstitial concentration of the peptide at arteriolar smooth muscle level. 3) There is substantial myocardial extraction of endothelin with coronary sinus levels lower than plasma levels, leaving unknown interstitial concentrations at which endothelin exerts its effects. 4) At the doses used, endothelin has no significant effects on systemic arterial pressure, cardiac output, or heart rate, despite significant decreases in coronary blood flow. 5) It was a major goal in the protocol to demonstrate a relation between total cumulative endothelin dose and the changes in resting perfusion defects after intravenous adenosine without making assumptions about interstitial concentrations extrapolated from plasma concentrations. 6) The selected model was designed to demonstrate an imaging concept, not to study the biology of endothelin and/or nitric oxide that is well known. Endothelin overexpression or imbalance with nitric oxide production may be only one of many potential vasomotor abnormalities causing resting vasoconstriction and perfusion abnormalities associated with endothelial dysfunction. Determining absolute myocardial perfusion in milliliters per gram per minute was also not essential for several reasons. 1) The relative coronary flow reserve, i.e., relative perfusion defects, is not dependent on heart rate and blood pressure changes whereas absolute coronary flow reserve using absolute flow measurements is highly dependent on changes in heart rate and blood pressure. 2) A review of 23 publications reporting absolute perfusion measurements by PET showed a great variability of 24% (SD 13) for rest and 29% (SD 12) for stress perfusion, expressed as the SD and mean value in milliliters per minute per gram. 3) Mathematical models for calculating absolute perfusion corrected for varying radionuclide extraction magnify differences in radionuclide uptake into greater differences in absolute perfusion that could obscure or exaggerate the differences observed. 4) The assumptions inherent in these mathematical models for calculating absolute myocardial perfusion are open to question for the relatively small but significant relative resting perfusion defects after intracoronary endothelin. 5) It was wished to demonstrate results using relative uptake values since accurate determination of the arterial input function required for models of absolute perfusion is technically difficult and makes PET data acquisition so complex that it introduces substantial variability and is rarely used in clinical practice. 6) Absolute quantification of myocardial perfusion also requires assumed corrections for partial volume errors that are equally questionable. 7) In this example, quantification of relative myocardial uptake is optimal for the specific hypothesis tested independent of varying heart rate and blood pressure and without the assumptions or model calculations on the primary data inherent in determining absolute perfusion.

This example demonstrates that intracoronary ET-1 causes visually apparent, quantitatively significant, long lasting resting myocardial perfusion defects by PET imaging that may persist or only partially improve after intravenous adenosine used for diagnostic imaging in the absence of myocardial scar and flow-limiting stenosis. These results may potentially explain in part the resting perfusion abnormalities on clinical PET images that may persist or only partially improve after adenosine stress and the resting perfusion heterogeneity by PET perfusion imaging associated with early nonobstructive coronary artery disease and endothelial dysfunction.

### Example 4

### Diagnostic Procedure for Detecting ET_{A} Receptor Mediated Coronary Microvascular Endothelial Dysfunction

Endothelin receptor A mediated coronary microvascular endothelial dysfunction is detected in an asymptomatic patient by (a) obtaining a first set of noninvasive cardiac PET perfusion images of a patient at rest and after dipyridamole or adenosine stress, as described in Example 1. (b) The resulting set of cardiac PET perfusion images are analyzed by applying Markovian homogeneity analysis, to obtain an initial myocardial perfusion homogeneity index for the patient. The Markovian homogeneity analysis is performed as described in Example 1. (c) Next, at least one selective endothelin receptor A antagonist is administered to the patient. The route of administration is preferably by mouth daily for one to two weeks or by intravenous injection of a pharmacologically acceptable solution of the antagonist. Examples of ET_{A}-receptor antagonists which may be used include, but are not limited to, Darusentan™ (Myogen), Sitaxsentan™ (Encysive), BQ123, BMS1822874, PD156707TTA101, 34-sulfatobastadin and BSF302146. The dosage amount of the antagonist and frequency of administration for a selected patient is determined using standard pharmacological procedures and applicable regulatory guidelines. (d) A second set of noninvasive cardiac PET perfusion images of the patient are obtained after administration of said antagonist, using the same PET scanning procedure as before at resting conditions and after dipyridamole or adenosine stress. (e) The second set of the cardiac PET perfusion images are analyzed by Markovian homogeneity analysis in the same manner as previously described, to obtain a second myocardial perfusion homogeneity index. (f) The first and second myocardial perfusion homogeneity indices are compared detect either an improvement of myocardial perfusion homogeneity or a lack of improvement. A result of improved myocardial perfusion homogeneity after administration of the antagonist indicates the presence of endothelin receptor A mediated coronary microvascular endothelial dysfunction in the patient.

Prior to administering the endothelin receptor A antagonist, a patient may exhibit a baseline resting myocardial perfusion homogeneity expressed as a Markovian homogeneity number that is outside about 2 standard deviation limits of a mean Markovian homogeneity number of a control group of normal healthy subjects. If the comparison of the patient's Markovian homogeneity indices reveals an increase in the Markovian homogeneity number after administration of the antagonist, then a diagnosis of ET_{A} receptor mediated coronary microvascular endothelial dysfunction is indicated. This result is also indicative of an elevated risk of coronary atherosclerosis, or future clinically manifest coronary artery disease, and increased risk of future coronary events. Such diagnostic information will aid the physician in establishing an appropriate preventative or treatment therapy for the patient directed toward improving coronary endothelial function and reducing risk factors. One such therapeutic treatment may include administering to the patient a myocardial perfusion homogeneity enhancing amount of one or more ET_{A} receptor antagonist. The diagnostic protocol may also include assessing whether the patient has one or more additional risk factors associated with coronary disease.

Analysis of a patient's cardiac PET perfusion images will preferably also detect any regional perfusion defects. Comparison of the patients' before and after PET perfusion images will indicate a reduction in the size and/or severity of one or more regional perfusion defects following administration of the endothelin receptor A antagonist. The analysis of the patient's cardiac PET perfusion images preferably also includes measurement of a base to apex longitudinal perfusion gradient. In some patients the gradient will be reduced following administration of the endothelin receptor A antagonist, indicating improvement in localized flow limiting stenosis following administration of the ET_{A} receptor antagonist.

For some patients, the comparison of cardiac PET perfusion images will reveal an abnormal baseline resting myocardial perfusion homogeneity, expressed as a Markovian homogeneity number, that is lower than the mean Markovian homogeneity number of a control group of healthy subjects by a margin greater than about 2 standard deviation limits. A result of no improvement of myocardial perfusion homogeneity after administration of the ET_{A}-receptor antagonist to a patient having a subnormal baseline homogeneity index indicates the absence of ET_{A} receptor mediated microvascular endothelial dysfunction in the patient. In this case, the absence of ET_{A} receptor mediated microvascular endothelial dysfunction may indicate other forms of early vascular disease that may indicate therapeutic treatment of the patient. A normal healthy individual without early vascular disease or microvascular endothelial dysfunction will have a Markovian homogeneity number, or index, that remains within the normal range of the control group after administration of the antagonist.

Without further elaboration, it is believed that one skilled in the art can, using the description herein, utilize the above-described apparatus and methods to their fullest extents. The foregoing embodiments are to be construed as illustrative, and not as constraining the remainder of the disclosure in any way whatsoever. Many variations and modifications of the embodiments disclosed herein are possible and are within the scope of the invention as defined by the appended claims. For example, Darusentan™ is considered to be representative of other ER_{A} antagonists that will provide similar or better myocardial perfusion homogeneity enhancing properties. Accordingly, the scope of protection is not limited by the description set out above, but is only limited by the claims which follow, that scope including all equivalents of the subject matter of the claims. The disclosures of all patents, patent applications and publications cited herein are hereby incorporated herein by reference, to the extent that they provide exemplary, procedural or other details supplementary to those set forth herein.

What is claimed is:

1. A method of detecting endothelin receptor A mediated coronary microvascular endothelial dysfunction in an asymptomatic subject, comprising:
(a) obtaining a first set of noninvasive cardiac PT perfusion images of the subject;
(b) analyzing the cardiac PET perfusion images obtained in step (a), wherein said analyzing comprises applying Markovian homogeneity analysis to said first set of images, to yield a first result comprising an initial myocardial perfusion homogeneity index;
(c) administering at least one selective endothelin receptor A antagonist to said subject;
(d) obtaining a second set of noninvasive cardiac PET perfusion images of said subject after said administration of said antagonist;
(e) analyzing the cardiac PET perfusion images obtained in step (d), wherein said analyzing comprises applying Markovian homogeneity analysis to said second set of images, to yield a second result comprising a second myocardial perfusion homogeneity index; and
(f) comparing said first and second results to detect improvement of myocardial perfusion homogeneity, or lack thereof, in said subject, wherein a result of improved myocardial perfusion homogeneity after administration of said antagonist indicates the presence of endothelin receptor A mediated coronary microvascular endothelial dysfunction in said subject.

2. The method of claim 1 wherein steps (a) and (d) further comprise administering a pharmacologic cardiac stress agent prior to obtaining said PET images.

3. The method of claim 2, wherein said cardiac stress agent comprises dipyridamole or adenosine, administered by intravenous infusion.

4. The method of claim 3 further comprising (g) comparing Markovian homogeneity analyses of cardiac PET perfusion images obtained with and without administration of said stress agent to said subject, wherein a result of improved myocardial perfusion homogeneity during pharmacologically induced cardiac stress is indicative of a stress perfusion abnormality.

5. The method of claim 1, wherein at least one endothelin receptor A antagonist is selected from the group consisting of Darusentan™, Sitaxsentan™, BQ123, BMS1822874, PD156707TTA101, 34-sulfatobastadin and BSF302146.

6. The method of claim 1, wherein improvement of myocardial perfusion homogeneity is quantitated at least in part by Markovian homogeneity analysis of cardiac PET perfusion images of the subject.

7. The method of claim 1, wherein the cardiac PET perfusion images comprise resting images.

8. The method of claim 1, wherein the subject, prior to administering the endothelin receptor A antagonist, exhibits a baseline resting myocardial perfusion homogeneity expressed as a Markovian homogeneity number that is outside about 2 standard deviation limits of a mean Markovian homogeneity number of a control group of normal healthy subjects.

9. The method of claim 8, wherein, following administration of the endothelin receptor A antagonist, an increase in the Markovian homogeneity number is obtained.

10. The method of claim 1, wherein the analysis of cardiac PET perfusion images comprises detection of regional perfusion defects, if any.

11. The method of claim 10, wherein, in (f), the comparison indicates a reduction in the size and/or severity of regional perfusion defects following administration of the endothelin receptor A antagonist.

12. The method of claim 1, wherein, in steps (b) and (e), the analysis of cardiac PET perfusion images comprises Markovian homogeneity analysis and either (i) observation of regional perfusion defects or (ii) measurement of a base to apex longitudinal perfusion gradient, or both (i) and (ii).

13. The method of claim 12, wherein the gradient is reduced following administration of the endothelin receptor A antagonist.

14. The method of claim 1, wherein in step (b) the analysis of the cardiac PET perfusion images reveals that an abnormal baseline resting myocardial perfusion homogeneity expressed as a Markovian homogeneity number that is lower than the mean Markovian homogeneity number of a control group of healthy subjects by a margin greater than about 2 standard deviation limits.

15. The method of claim 1 wherein, in step (f), a result of no improvement of myocardial perfusion homogeneity after said administration of said ET_{A}-receptor antagonist indicates the absence of ET_{A}-mediated microvascular endothelial dysfunction in said subject.

16. The method of claim 15, wherein said indication of an absence of endothelin receptor A mediated microvascular endothelial dysfunction in the subject is diagnostic of early coronary artery disease.

17. The method of claim 16, wherein the diagnosis of early coronary artery disease indicates therapeutic treatment of said subject to improve coronary endothelial function and/or to reduce coronary artery disease risk factors.

18. The method of claim 17 wherein one said therapeutic treatment comprises administration of a myocardial perfusion homogeneity enhancing amount of at least one endothelin receptor A antagonist.

19. The method of claim 1, wherein the subject has one or more risk factors associated with coronary disease.

20. The method of claim 1, wherein an indication of the presence of endothelin receptor A mediated microvascular endothelial dysfunction is diagnostic of existing coronary atherosclerosis and/or elevated risk of future coronary artery disease in the subject.
